(19) United States
(12) Patent Application Publication
(10) Pub. No.: US 2004/0101528 A1
(43) Pub. Date: May 27, 2004

(54) Type I IL-1 receptors

(76) Inventors: Steven K. Dower, Redmond, WA (US); Carl J. March, Winslow, WA (US); John E. Sims, Seattle, WA (US); David L. Urdal, Seattle, WA (US)

Correspondence Address: IMMUNEX CORPORATION, LAW DEPARTMENT, 1201 AMGEN COURT WEST, SEATTLE, WA, 98119, US

(21) Appl. No.: 10/309,512

(22) Filed: Dec. 3, 2002

Related U.S. Application Data
(63) Continuation of application No. 08/452,775, filed on May 30, 1995, now Pat. No. 6,511,665.
Application No. 08/452,775 is a continuation of application No. 07/575,911, filed on Aug. 31, 1990, now abandoned.
Application No. 07/575,911 is a continuation-in-part of application No. 07/258,756, filed on Oct. 13, 1988, now Pat. No. 5,081,228.
Application No. 07/258,756 is a continuation-in-part of application No. 07/160,550, filed on Feb. 25, 1988, now Pat. No. 4,968,607.

(51) Int. Cl. A61K 39/395; C07K 16/24

(52) U.S. Cl. 424/145.1; 530/388.23

(57) ABSTRACT

The present invention provides monoclonal antibodies and binding proteins which specifically bind to the IL-1 receptor. Also provided are methods for detecting IL-1 receptors on cells, and for detecting soluble IL-1 receptors in serum.

### CROSS-REFERENCE TO RELATED APPLICATION

This application is a continuation-in-part of pending U.S. application Ser. No. 07/258,756, filed Oct. 13, 1988, which is a continuation-in-part of pending U.S. application Ser. No. 07/160,550, filed Feb. 25, 1988, which is a continuation-in-part of pending U.S. application Ser. No. 07/125,627, filed Nov. 25, 1987.

### TECHNICAL FIELD

The present invention relates generally to antibodies and, more specifically, to antibodies against interleukin-1 receptors.

### BACKGROUND OF THE INVENTION

Interleukin-1α and Interleukin-1β (IL-1α and IL-1β) are distantly related polypeptide hormones which play a central role in the regulation of immune and inflammatory responses. These two proteins were originally both classified as IL-1, based on a shared lymphocyte activation factor (LAF) activity, and a common major cellular source, activated macrophages. As information has accumulated from studies using purified natural and recombinant IL-1 molecules, it has become clear that IL-1α and IL-1β each mediate most, if not all, of the wide range of activities previously ascribed to IL-1. The basis for this nearly identical spectrum of biological activities is thought to be a single class of plasma membrane IL-1 receptors which bind both IL-1α and IL-1β. The binding of IL-1 to these receptors is specific, and occurs with a high affinity (1×10⁻¹⁰M).

Polyclonal antibodies have long been utilized for various aspects of research and development, but in 1975 Köhler and Milstein discovered a new technique which revolutionized the production and use of antibodies (see Köhler and Milstein, Nature 256:495 1975). This technique utilized somatic cell hybridization to generate a continuous “hybridoma” cell line which produced large quantities of a single specific antibody, also referred to as a monoclonal antibody.

It would be beneficial if such antibodies against the IL-1 receptor were available, as they may be useful for diagnosis and therapy, as well as for various research applications. For example, antibodies may be utilized in clinical applications to diagnose the presence of IL-1 receptor in a patient's serum, or may be administered therapeutically to bind to and target IL-1 receptor bearing cells for elimination or neutralization. Additionally, antibodies may be utilized in various research applications such as the purification of recombinantly produced IL-1 receptor, or in assays which detect the presence of the IL-1 receptor.

The present invention provides such antibodies and, furthermore, provides other related advantages.

### SUMMARY OF THE INVENTION

The present invention provides monoclonal antibodies which specifically bind to mammalian IL-1 receptors. Within one embodiment of the invention, the monoclonal antibody is selected from the group consisting of human and mouse monoclonal antibodies. Within selected embodiments, the monoclonal antibody blocks the binding of IL-1 to the IL-1 receptor. Within another embodiment the mammalian IL-1 receptor is selected from the group consisting of murine and human IL-1 receptor. Within a related aspect, a therapeutic composition is provided comprising a monoclonal antibody to the IL-1 receptor as described above and a physiologically acceptable carrier or diluent.

Within another aspect of the present invention, a binding protein is provided which specifically binds to a mammalian IL-1 receptor, which may be, for example, a fragment of an antibody or a fusion protein comprising at least one domain derived from an antibody. Within a related aspect, a therapeutic composition is provided comprising a binding protein which specifically binds to mammalian IL-1 receptor, and a physiologically acceptable carrier or diluent.

Within yet another aspect of the present invention, a method for detecting IL-1 receptors on cells is provided comprising the steps of (a) incubating the cells with a monoclonal antibody, as described above, which is labeled, and (b) detecting the presence of bound antibody. Within another aspect, a method for detecting soluble IL-1 receptor in serum is provided comprising the steps of (a) incubating serum suspected of containing soluble IL-1 receptor with a solid support having monoclonal antibodies as described above affixed thereto under conditions and for a time sufficient for binding to occur, (b) incubating the solid support with a second labeled monoclonal antibody specific for mammalian IL-1 receptors under conditions and for a time sufficient for binding to occur, and (c) detecting the presence of bound labeled antibody.

These and other aspects of the present invention will become evident upon reference to the following detailed description and attached drawings.

### BRIEF DESCRIPTION OF THE DRAWINGS

FIG. 1 is a restriction map of cDNA constructs comprising the coding regions of the murine and human IL-1R genes. The murine fragment, isolated from EL-4 6.1 C10 cells and present as an insert in clone GEMBL78, has been deposited with the American Type Culture Collection under deposit accession no. ATCC 67563.

FIG. 2 depicts the cDNA sequence of clone GEMBL78. Nucleotides are numbered from the beginning of the fragment. The CTG codon specifying the leucine residue constituting the N-terminus is underlined at position 282, and the TAG terminator codon which ends the open reading frame is underlined at position 1953.

FIGS. 3A-3C depict the cDNA sequence and derived amino acid sequence of the coding region of the cDNA shown in FIG. 2. In FIGS. 3A-3C, nucleotides and amino acids are numbered from the leucine residue representing the N-terminus of the mature protein. In FIGS. 3A-3C, the alternative initiator methionines, N-terminus, and 21 amino acid putative transmembrane region of the murine IL-1 receptor are underlined.

FIG. 4 depicts a cDNA sequence which includes the complete coding region of the human IL-1R gene. Nucleotides are numbered from the beginning of a fragment, designated R3A, which includes the N-terminus and a short sequence of 5′ nontranslated DNA. The CTG codon specifying the leucine residue constituting the N-terminus is underlined at position 135, and the TAG terminator codon which ends the open reading frame is underlined at position 1791.

FIGS. 5A-5C depict the cDNA sequence and derived amino acid sequence of the coding region of a cDNA encoding human IL-1 receptor. In FIGS. 5A-5C, nucleotides and amino acids are numbered from the leucine residue (underlined) representing the N-terminus of the mature protein. The 20-amino acid transmembrane region is also underlined.

FIG. 6 is a schematic illustration of the mammalian high expression plasmid pCD201, which is described in greater detail in Example 6.

FIG. 7 provides a graphical comparison of the IL-1 binding characteristics of natural and recombinant IL-1 receptors. FIG. 7A compares direct binding of ¹²⁵I-IL-1α to cells expression native IL-1 receptor (EL-4 6.1 C10) or recombinant receptor (CIS-IL-1R0). FIG. 7B shows the data from FIG. 7A) replotted in the Scatchard coordinate system. FIG. 7C indicates competition for ¹²⁵I-IL-1α binding by unlabeled IL-1α and IL-1β. In FIG. 7, C indicates the concentration of IL-1 added to the binding incubation (molar); r indicates molecules of IL-1 bound per cell.

FIG. 8 is a comparison of the derived amino acid sequences of the murine and human IL-1 receptors. The transmembrane regions of each protein are underlined, and conserved cysteine residues are indicated by asterisks. Potential N-linked glycosylation sites are indicated by triangles adjacent to asparagine residues.

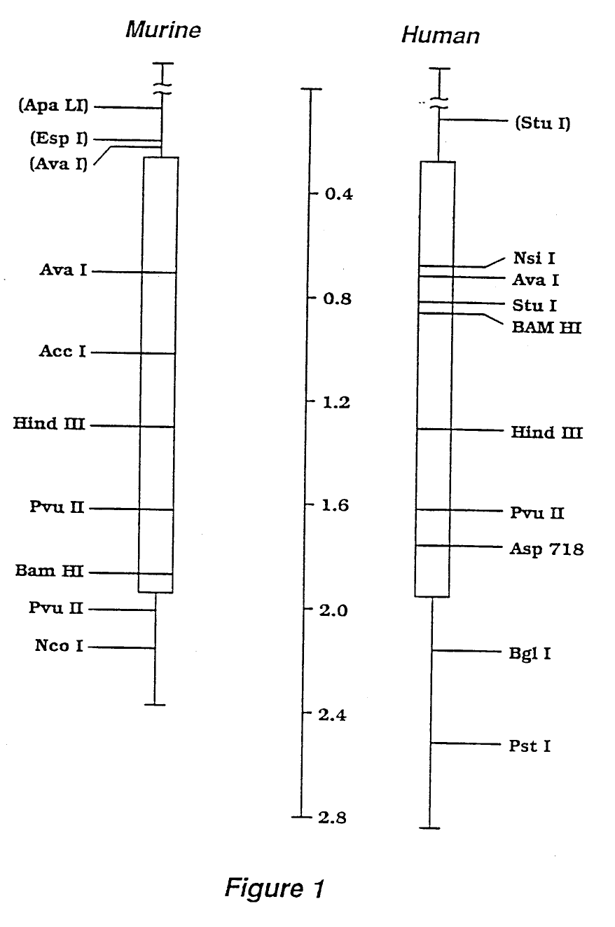

### DETAILED DESCRIPTION OF THE INVENTION

IL-1α and IL-1β apparently regulate the metabolism of cells through a common plasma membrane receptor protein. IL-1 receptor from detergent solutions of EL-4 6.1 C10 cells has been stably adsorbed to nitrocellulose with full retention of IL-1 binding activity. This assay system was used to monitor the purification of the IL-1 receptor and to investigate the effects of several chemical modifications on receptor binding activity. IL-1 receptors extracted from EL-4 6.1 C10 cells can be bound to and specifically eluted from IL-1α coupled to Sepharose or other suitable affinity chromatography supports.

Purification by the foregoing process resulted in the identification by silver staining of polyacrylamide gels of a protein of Mᵣ 82,000 daltons that was present in fractions exhibiting IL-1 binding activity. Experiments in which the cell surface proteins of EL-4 cells were radiolabeled and ¹²⁵I labeled receptor was purified by affinity chromatography suggested that the Mᵣ 82,000 protein was expressed on the plasma membrane. N-glycanase treatment of this material showed that 21%-35% of the total Mᵣ (82,000) of the receptor was N-lined carbohydrate.

In order to define the chemical properties of the IL-1 receptor, a simple, reproducible and quantitative assay system was devised for the detection of IL-1 receptor in detergent solutions. With this assay, receptor purification can be followed, and changes in receptor binding activity in response to chemical modification of the receptor can be easily monitored.

### Binding Assay for IL-1 Receptor

Recombinant human IL-1β and IL-1α can be prepared by expression in E. coli and purification to homogeneity as described by Kronheim et al. (Bio/Technology 4:1078, 1986). Recombinant human IL-1α is preferably expressed as a polypeptide composed of the C-terminal 157 residues of IL-1α, which corresponds to the Mᵣ 17,500 form of the protein released by activated macrophages. The purified protein is stored at −70° C. in phosphate buffered saline as a stock solution of 3 mg/ml. 10 μl (30 μg) aliquots of the stock solution are labeled with sodium (¹²⁵I) iodide by a modified chloramine-T method described by Dower et al. (Nature 324:266, 1986) and Segal et al. (J. Immunol. 118:1338, 1977). In this procedure, 10 μg rIL-1α (0.57 nmol) in 10 μl phosphate (0.05 M) buffered saline (0.15 M) pH 7.2 (PBS) are added to 2.5 mCi (1.0 nmol) of sodium iodide in 25 μl of 0.05 M sodium phosphate pH 7.0. The reaction is initiated by addition of 30 μl of 1.4×10-4 M chloramine-T (4.2 mmol; Sigma Chemical Co., St. Louis, Mo.). After 30 minutes on ice the reaction mixture is fractionated by gel filtration on a 1 ml bed volume Biogel P6 (Bio-Rad, Richmond, Calif.) column. Routinely, 40%-50% of ¹²⁵I is incorporated into protein.

¹²⁵I-IL-1α can be purified by gel filtration or other suitable methods and immediately diluted to a working stock solution of 3×10⁻⁸ M in Roswell Park Memorial Institute (RPMI) 1640 medium comprising 1% (w/v) bovine serum albumin (BSA), 0.1% (w/v) sodium azide, 20 mM Hepes pH 7.4 (binding medium), to avoid radiolysis. Such dilute solutions can be stored for up to one month without detectable loss of receptor binding activity. The specific activity is routinely in the range 1-3×10¹⁵ cpm/mmole (ca 1 atom of iodine per IL-1α molecule). Typically, the labeled protein is initially (prior to dilution) 100% active as determined by its capacity to elicit IL-2 production from EL-4 6.1 C10 cells. Further, 100% of the ¹²⁵I cpm can be precipitated by trichloroacetic acid and >95% can be absorbed by IL-1 receptor bearing cells.

EL-4 6.1 C10 cells are propagated in suspension culture as described by MacDonald et al., J. Immunol. 135:3964, 1985. An IL-1 receptor negative variant line of EL-4 cells, EL-4 (M) (ATCC TIB 39), is grown in an identical fashion. Cells are monitored on a weekly basis for IL-1 receptor expression by ¹²⁵I-IL-1α binding.

To maintain relatively high levels of receptor expression, cells can be sorted using fluorescence-activated cell sorting (FACS) and fluorescein-conjugated recombinant IL-1α. Fluorescein-conjugated rIL-1α (FITC IL-1α) is prepared by reacting 2.9 nanomoles protein with 100 nanomoles of fluorescein isothiocyanate (Research Organics, Cleveland, Ohio) in a total volume of 70 μl of borate (0.02 M) buffered saline (0.15 M) pH 8.5 for two hours at 37° C. Protein is separated from unconjugated dye by gel filtration on a 1 ml bed volume P6 column, as described by Dower et al. (J. Exp. Med 162;501, 1985). Using an EPICS C flow cytometer (Coulter Instruments; 488 nM argon laser line, 300 MW, gain 20, PMT voltage 1700), cells providing the highest level fluorescence signal (e.g., the top 1.0% or 0.1%, as desired) are collected and used to establish cell cultures for receptor expression.

For extractions, cells harvested from culture by centrifugation are washed once with binding medium and sedimented at 2000× g for 10 min. to form a packed pellet (ca 8×10⁸ cells/ml). To the pellet is added an equal volume of PBS containing 1% Triton X-100 and a cocktail of protease inhibitors (2 mM phenylmethylsulphonyl fluoride, 1 μM pepstatin, 1 μM leupeptin, and 2 mM O-phenanthroline). The cells are mixed with the extraction buffer by vigorous vortexing and the mixture incubated on ice for 15 minutes; at the end of this time the mixture is centrifuged at 11,000×g for 30 minutes at 8° C. to remove nuclei and other debris. The supernatant is made 0.02% w/v in sodium azide and stored either at 8° C. or −70° C., with no loss in IL1 receptor activity detected for periods of up to six months at either temperature.

For solid phase binding assays, unless otherwise indicated, 1 μl (4×10⁵ cell equivalents) aliquots of extract are placed on dry BA85/21 nitrocellulose membranes (Schleicher & Schuell, Keene, N.H.) and the membranes kept at room temperature until dry. Dry membranes can be stored at room temperature until use. Under these conditions, receptor binding activity remains stable for up to two months. Prior to use, membranes are reconstituted by incubating for 30 minutes in Tris (0.05 M) buffered saline (0.15 M) pH 7.5 containing 3% w/v BSA to block nonspecific binding sites, washed twice with PBS (20 ml per filter), once with binding medium and cut while wet into 0.9×0.9 cm squares with the IL-1 receptor extract at the center. The squares are placed in 24 well trays (Costar, Cambridge, Mass.) and covered with 200 μl of binding medium containing ¹²⁵I-IL-1α or ¹²⁵I-IL-1α and unlabeled inhibitors. Trays are then placed on a nutator and incubated in a refrigerator (8° C.) for two hours. At the end of this time a 60 μl aliquot can be taken from each well for determination of unbound ¹²⁵I-rIL-1α. Subsequently, the remaining solution is aspirated and discarded and the nitrocellulose filters washed by adding and aspirating sequentially 1 ml of binding medium and three times 1 ml of PBS to each well. The nitrocellulose squares are then removed and dried on filter paper. Subsequently, they are either placed on Kodak X-Omat AR film for twelve hours at −70° C., or placed in 12×75 cm glass tubes and counted on a gamma counter.

### Definitions

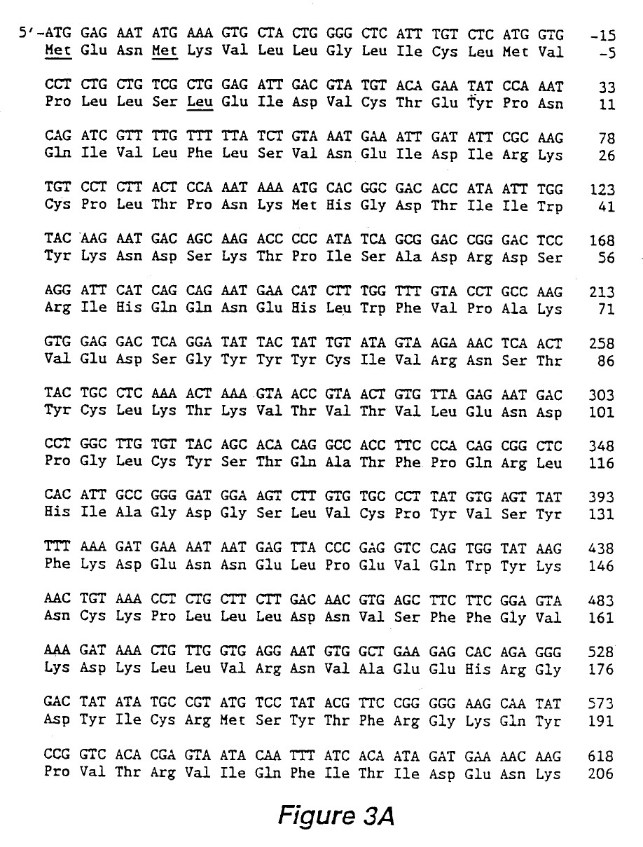

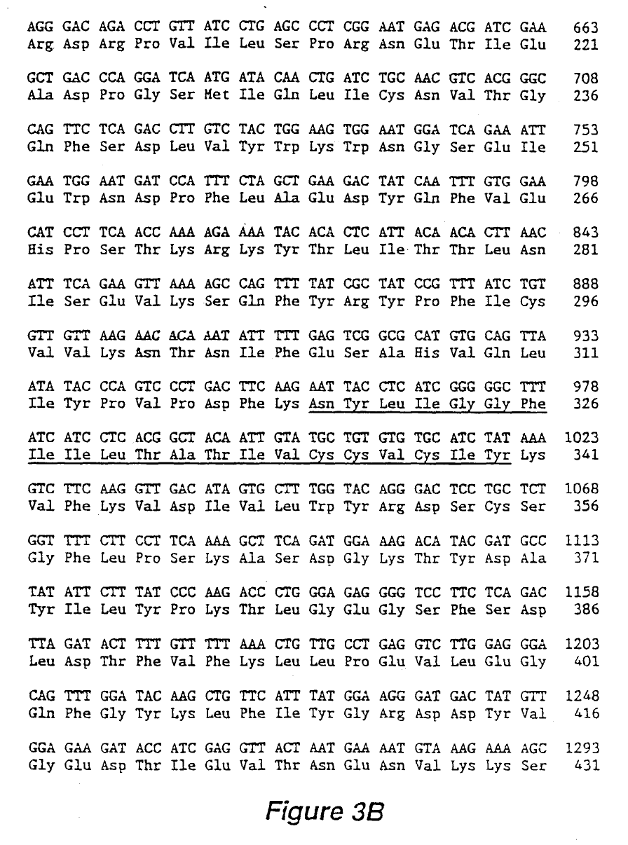

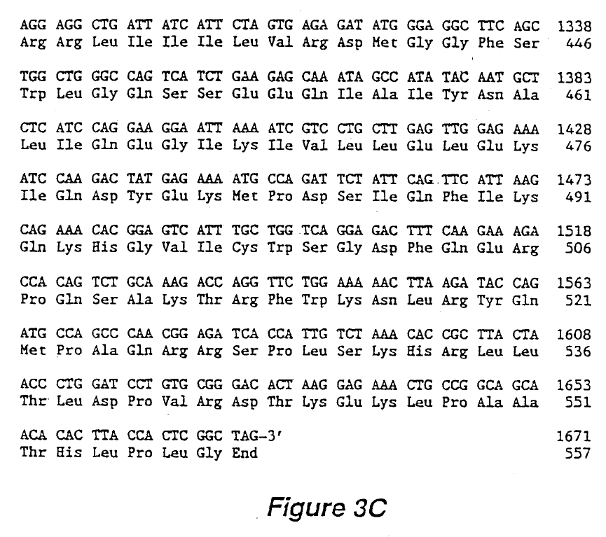

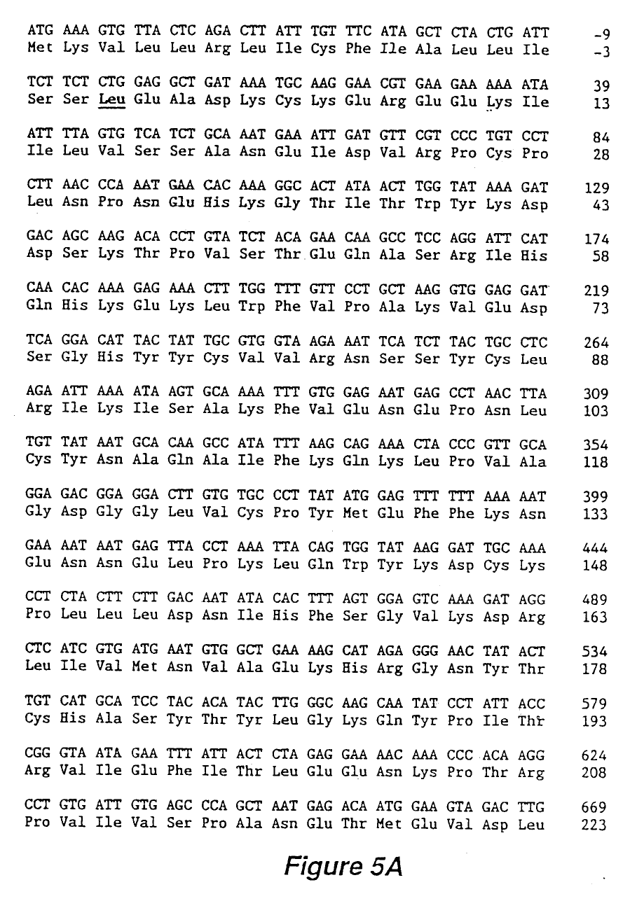

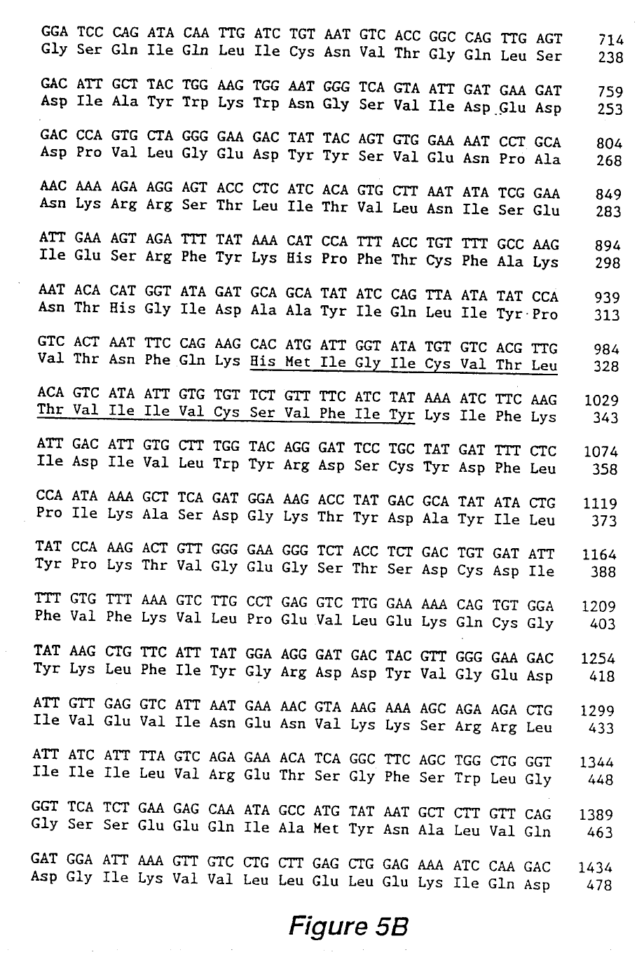

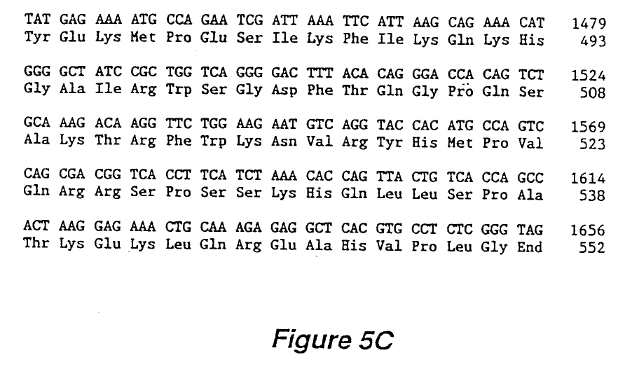

“Interleukin-1 receptor” and “IL-1R” refer to proteins which are capable of binding Interleukin-1 (L1) molecules and, in their native configuration as mammalian plasma membrane proteins, presumably play a role in transducing the signal provided by IL-1 to a cell. As used herein, the term includes analogs of native proteins with IL-1 binding or signal transducing activity. Specifically included are truncated or soluble forms of the IL-1 receptor protein not having a cytoplasmic and transmembrane region. The predicted molecular weight of the murine protein corresponding to the sequence of the mature protein depicted in FIGS. 3A-3B is 64,597 daltons, while the predicted weight of the precursor is 66,697 daltons. Both of these estimates are exclusive of any glycosylation. The predicted molecular weight of the human protein corresponding to the sequence of the mature protein depicted in FIGS. 5A-5C is 63,486 daltons, while the predicted weight of the precursor is 65,402 daltons.

“Recombinant,” as used herein, means that a protein is derived from recombinant (e.g., microbial or mammalian) expression systems. “Microbial” refers to recombinant proteins made in bacterial or fungal (e.g. yeast) expression systems. As a product, “recombinant microbial” defines a protein essentially free of native endogenous substances and unaccompanied by associated native glycosylation. Protein expressed in most bacterial cultures, e.g. E. coli, will be free of glycan; protein expressed in yeast may have a glycosylation pattern different from that expressed in mammalian cells.

“Biologically active,” as used throughout the specification as a characteristic of IL-1 receptors, means either that a particular molecule shares sufficient amino acid sequence similarity with the embodiments of the present invention disclosed herein to be capable of binding at least 0.01 nmoles IL-1 per nanomole IL-1 receptor or IL-1 receptor analog, or, in the alternative, shares sufficient amino acid sequence similarity to be capable of transmitting an IL-1 stimulus to a cell, for example, as a component of a hybrid receptor construct. Preferably, biologically active IL-1 receptors within the scope of the present invention are capable of binding greater than 0.1 nanomoles IL-1 per nanomole receptor, and most preferably, greater than 0.5 nanomoles IL-1 per nanomole receptor.

“DNA sequence” refers to a DNA polymer, in the form of a separate fragment or as a component of a larger DNA construct, which has been derived from DNA isolated at least once in substantially pure form, i.e., free of contaminating endogenous materials and in a quantity or concentration enabling identification, manipulation, and recovery of the sequence and its component nucleotide sequences by standard biochemical methods; for example, using a cloning vector. Such sequences are preferably provided in the form of an open reading frame uninterrupted by internal nontranslated sequences, or introns, which are typically present in eukaryotic genes. However, it will be evident that genomic DNA containing the relevant sequences could also be used. Sequences of non-translated DNA may be present 5′ or 3′ from the open reading frame, where the same do not interfere with manipulation or expression of the coding regions.

“Nucleotide sequence” refers to a heteropolymer of deoxyribonucleotides. DNA sequences encoding the proteins provided by this invention are assembled from cDNA fragments and short oligonucleotide linkers, or from a series of oligonucleotides, to provide a synthetic gene which is capable of being expressed in a recombinant transcriptional unit.

“Recombinant expression vector” refers to a plasmid comprising a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate transcription and translation initiation and termination sequences. Structural elements intended for use in yeast expression systems preferably include a leader sequence enabling extracellular secretion of translated protein by a host cell. Alternatively, where recombinant protein is expressed without a leader or transport sequence, it may include an N-terminal methionine residue. This residue may optionally be subsequently cleaved from the expressed recombinant protein to provide a final product.

“Recombinant microbial expression system” means a substantially homogeneous monoculture of suitable host microorganisms, for example, bacteria such as E. coli or yeast such as S. cerevisiae, which have stably integrated a recombinant transcriptional unit into chromosomal DNA or carry the recombinant transcriptional unit as a component of a resident plasmid. Generally, cells constituting the system are the progeny of a single ancestral transformant. Recombinant expression systems as defined herein will express heterologous protein upon induction of the regulatory elements linked to the DNA sequence or synthetic gene to be expressed.

### ISOLATION OF cDNAs ENCODING IL-1 RECEPTORS

In order to secure the murine coding sequence, a DNA sequence encoding murine IL-1R (mIL-1R) was isolated from a cDNA library prepared by reverse transcription of polyadenylated RNA isolated from the murine cell line EL-4 6.1 C10. The library was screened by direct expression of pooled cDNA fragments in monkey COS-7 cells using a mammalian expression vector (pDC201) that uses regulatory sequences derived from SV40 and Adenovirus 2. Transfectants expressing biologically active IL-1R were identified by incubating transfected COS-7 cells with medium containing ¹²⁵I-IL-1α, washing the cells to remove unbound labeled IL-1α, and contacting the cell monolayers with X-ray film to detect concentrations of IL-1α binding. Transfectants detected in this manner appear as dark foci against a relatively light background.

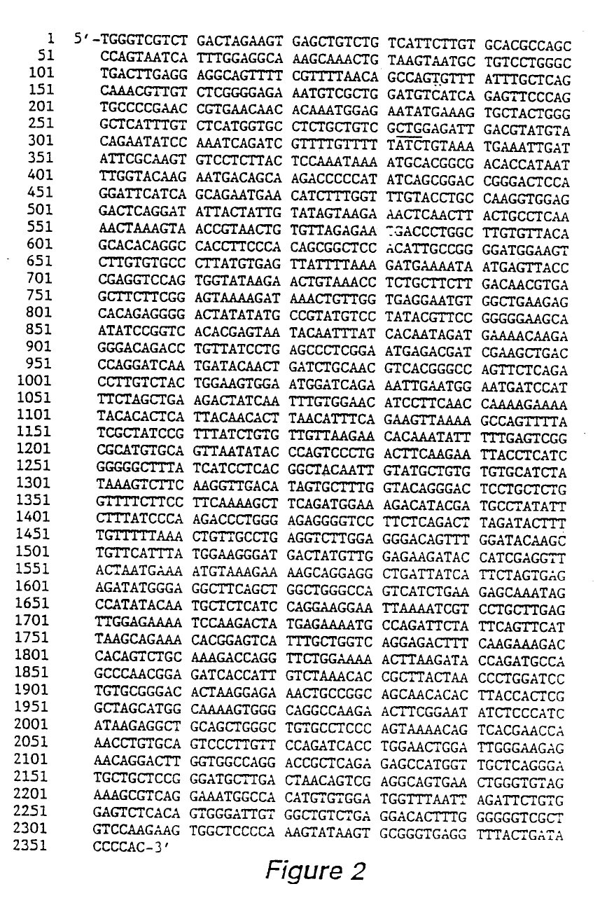

Using this approach, approximately 150,000 cDNAs were screened in pools of approximately 350 cDNAs until assay of one transfectant pool indicated positive foci of IL-1α binding. A frozen stock of bacteria from this positive pool was grown in culture and plated to provide individual colonies, which were screened until a single clone (clone 78) was identified which directed synthesis of a surface protein with detectable IL-1 binding activity. This clone was isolated, and its insert sequenced to determine the sequence of the murine cDNA set forth in FIG. 2. The initiator methionine for the full-length translation product of the native murine gene is one of two alternative methionine residues found at positions −19 and −16 of FIG. 3A. The first amino acid residue of the mature receptor protein was deduced by comparison to an N-terminal amino acid sequence obtained from highly purified preparations of IL-1R derived from EL-4 6.1 C10 cells. This residue is a leucine residue shown at position 1 of FIG. 3A. The 1671 nucleotide coding region corresponding to the mature protein encodes 576 amino acids, including 15 cysteine residues and a 21-amino acid putative transmembrane region. Located N-terminal to the transmembrane region are 7 potential N-glycosylation sites. A cloning vector comprising the full-length murine cDNA, designated GEMBL78, has been deposited with the American Type Culture Collection (ATCC), Rockville, Md., under accession no. 67563. The deposit was made under the conditions of the Budapest Treaty.

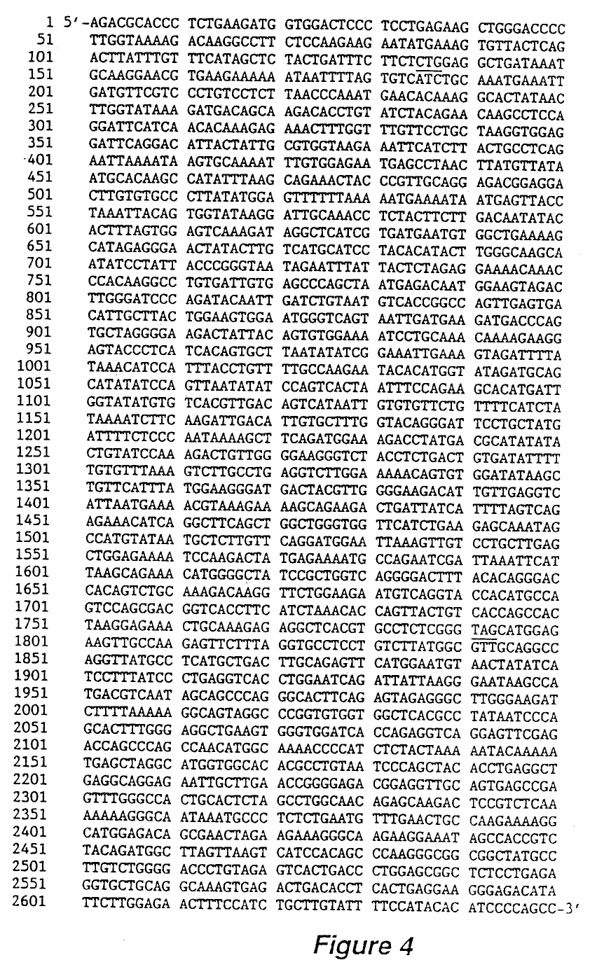

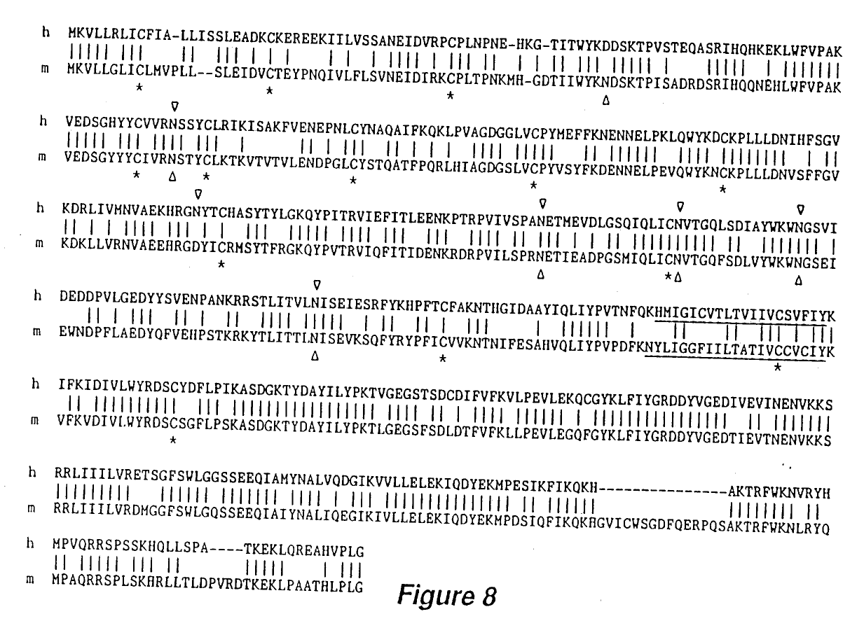

A probe was constructed from the murine sequence and used to screen human cDNA libraries prepared from cultures of a human T-cell clone grown in the presence of OKT3 antibody and IL-2. cDNA clones which hybridized to the murine probe were then isolated and sequenced. Using a fragment derived from human cDNA clones, a 1707 nucleotide human coding sequence was obtained and sequenced. The nucleotide sequence of the human cDNA, including 5′ and 3′ nontranslated sequences, is shown in FIG. 4. The nucleotide sequence of the human open reading frame and derived amino acid sequence of the human protein is set forth in FIGS. 5A-5C. This sequence comprises 569 amino acids (including a 17 amino acid signal peptide), including 16 cysteine residues, 13 of which are conserved between the murine and human genes. In addition, the human sequence includes six potential N-glycosylation sites, of which 5 are conserved between murine and human. The amino acid sequence of FIGS. 5A-5C is numbered from a leucine residue considered to be the likely N-terminus on the basis of comparison to the murine protein. The putative transmembrane region of the human gene is 20 amino acids in length. The sequences of the presumed intracellular portions of the murine and human genes are highly (87%) conserved; the extracellular (78%) and transmembrane regions (63%) are somewhat less conserved, except for the location of cysteines presumably involved in intramolecular disulfide bonding and certain N-glycosylation sites. The derived amino acid sequences of the human and murine genes are compared in FIG. 8.

The murine and human genes encode integral membrane proteins including intracellular regions having no apparent homology with any known protein sequence and extracellular portions which appear to be organized into domains similar to those of members of the immunoglobulin gene superfamily. Immunoglobulin-like domains typically possess only minimal amino acid similarity but share a common three-dimensional structure consisting of two β-sheets held together by a disulfide bond. The cysteine residues involved in formation of this disulfide bond, as well as a few other critical residues, are highly conserved and occur in the same relative position in almost all members of the family. Members of the immunoglobulin superfamily include not only immunoglobulin constant and variable regions but also a number of other cell surface molecules, many of which are involved in cell-cell interactions.

Like most mammalian genes, mammalian IL-1Rs are presumably encoded by multi-exon genes. Alternative mRNA constructs which can be attributed to different mRNA splicing events following transcription, and which share large regions of identity or similarity with the cDNAs claimed herein, are considered to be within the scope of the present invention.

In its nucleic acid embodiments, the present invention provides DNA sequences encoding mammalian IL-1Rs. Examples of mammalian IL-1Rs include primate IL-1R, human IL-1R, murine, canine, feline, bovine, ovine, equine and porcine IL-1Rs. IL-1R DNAs are preferably provided in a form which is capable of being expressed in a recombinant transcriptional unit under the control of mammalian, microbial, or viral transcriptional or translational control elements. For example, a sequence to be expressed in a microorganism will contain no introns. In preferred aspects, the DNA sequences comprise at least one, but optionally more than one sequence component derived from a cDNA sequence or copy thereof. Such sequences may be linked or flanked by DNA sequences prepared by assembly of synthetic oligonucleotides. However, synthetic genes assembled exclusively from oligonucleotides could be constructed using the sequence information provided herein. Exemplary sequences include those substantially identical to the nucleotide sequences depicted in FIGS. 3A-3C. Alternatively, the coding sequences may include codons encoding one or more additional amino acids located at the N-terminus, for example, an N-terminal ATG codon specifying methionine linked in reading frame with the nucleotide sequence. Due to code degeneracy, there can be considerable variation in nucleotide sequences encoding the same amino acid sequence; exemplary DNA embodiments are those corresponding to the sequence of nucleotides 1-1671 of FIGS. 3A-3C, and nucleotides 1-1656 of FIGS. 5A-5C. Other embodiments include sequences capable of hybridizing to the sequence of FIGS. 3A-3C or 5A-5C under moderately stringent conditions (50° C., 2×SSC) and other sequences degenerate to those described above which encode biologically active IL-1R polypeptides.

The present invention also provides expression vectors for producing useful quantities of purified IL-1R. The vectors can comprise synthetic or cDNA-derived DNA fragments encoding mammalian IL-1Rs or bioequivalent homologues operably linked to regulatory elements derived from mammalian, bacterial, yeast, bacteriophage, or viral genes. Useful regulatory elements are described in greater detail below. Following transformation, transfection or infection of appropriate cell lines, such vectors can be induced to express recombinant protein.

Mammalian IL-1Rs can be expressed in mammalian cells, yeast, bacteria, or other cells under the control of appropriate promoters. Cell-free translation systems could also be employed to produce mammalian IL-1R using RNAs derived from the DNA constructs of the present invention. Appropriate cloning and recombinant expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference.

Various mammalian cell culture systems can be employed to express recombinant protein. Examples of suitable mammalian host cell lines include the COS-7 lines of monkey kidney cells, described by Gluzman (Cell 23:175, 1981), and other cell lines capable of expressing an appropriate vector, for example, the C127, 3T3, CHO, HeLa and BHK cell lines. Mammalian expression vectors may comprise nontranscribed elements such as an origin of replication, a suitable promoter and enhancer, and other 5′ or 3′ flanking nontranscribed sequences, and 5′ or 3′ nontranslated sequences, such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Additional details regarding the use of a mammalian high expression vector to produce a recombinant mammalian IL-1R are provided in Examples 4 and 6, below. Exemplary vectors can be constructed as disclosed by Okayama and Berg (Mol. Cell. Biol. 3:280, 1983).

A useful system for stable high level expression of mammalian receptor cDNAs in C127 murine mammary epithelial cells can be constructed substantially as described by Cosman et al. (Molecular Immunol. 23:935, 1986).

Yeast systems, preferably employing Saccharomyces species such as S. cerevisiae, can also be employed for expression of the recombinant proteins of this invention. Yeast of other genera, for example, Pichia or Kluyveromyces, have also been employed as production strains for recombinant proteins.

Useful recombinant expression vectors for bacterial use are constructed by inserting a DNA sequence encoding mammalian IL-1R together with suitable translation initiation and termination signals in operable reading phase with a functional promoter. The vector will comprise one or more phenotypic selectable markers and an origin of replication to ensure amplification within the host. Suitable prokaryotic hosts for transformation include E. coli, Bacillus subtilis, Salmonella typhimurium, and various species within the genera Pseudomonas, Streptomyces, and Staphylococcus, although others may also be employed as a matter of choice.

Expression vectors are conveniently constructed by cleavage of cDNA clones at sites close to the codon encoding the N-terminal residue of the mature protein. Synthetic oligonucleotides can then be used to “add back” any deleted sections of the coding region and to provide a linking sequence for ligation of the coding fragment in appropriate reading frame in the expression vector, and optionally a codon specifying an initiator methionine.

Preferably, purified mammalian IL-1Rs or bioequivalent homologues are prepared by culturing suitable host/vector systems to express the recombinant translation products of the synthetic genes of the present invention, which are then purified from culture media

An alternative process for producing purified IL-1R involves purification from cell culture supernatants or extracts. In this approach, a cell line which elaborates useful quantities of the protein is employed. Supernatants from such cell lines can be optionally concentrated using a commercially available protein concentration filter, for example, an Amicon or Millipore Pellicon ultrafiltration unit. Following the concentration step, the concentrate can be applied to a suitable purification matrix as previously described. For example, a suitable affinity matrix can comprise an IL-1 or lectin or antibody molecule bound to a suitable support. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred.

Finally, one or more reversed-phase high performance liquid chromatography (RP-HPLC) steps employing hydrophobic RP-HPLC media, e.g., silica gel having pendant methyl or other aliphatic groups, can be employed to further purify an IL-1R composition. Some or all of the foregoing purification steps, in various combinations, can also be employed to provide a homogeneous recombinant protein.

Recombinant protein produced in bacterial culture is usually isolated by initial extraction from cell pellets, followed by one or more concentration, salting-out, aqueous ion exchange or size exclusion chromatography steps. Finally, high performance liquid chromatography (HPLC) can be employed for final purification steps. Microbial cells employed in expression of recombinant mammalian IL-1R can be disrupted by any convenient method, including freeze-thaw cycling, sonication, mechanical disruption, or use of cell lysing agents.

Fermentation of yeast which express mammalian IL-1R as a secreted protein greatly simplifies purification. Secreted recombinant protein resulting from a large-scale fermentation can be purified by methods analogous to those disclosed by Urdal et al. (J. Chromatog. 296:171, 1984). This reference describes two sequential, reversed-phase HPLC steps for purification of recombinant human GM-CSF on a preparative HPLC column. Numerous DNA constructions including all or part of the nucleotide sequences depicted in FIGS. 3A-3C or 5A-5C, in conjunction with oligonucleotide cassettes comprising additional useful restriction sites, can be prepared as a matter of convenience. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene having particular codons altered according to the substitution, deletion, or insertion required. By way of example, Walder et al. (Gene 42:133, 1986); Bauer et al. (Gene 37:73, 1985); Craik (Biotechniques, pp. 12-19, 1985); Smith et al. (Genetic Engineering. Principles and Methods, Plenum Press, 1981); and U.S. Pat. No. 4,518,584 discloses suitable techniques, and are incorporated by reference herein.

### Antibodies

Purified IL-1 receptor may be utilized to prepare both monoclonal and polyclonal antibodies, as well as other binding proteins which may be specifically constructed utilizing recombinant DNA methods. These binding proteins incorporate the variable regions from a gene which encodes a specifically binding monoclonal antibody. Within the context of the present invention, monoclonal antibodies and binding proteins are defined to be specifically binding if they bind with a Kₐ of greater than or equal to 10⁷ M⁻¹. The affinity of a monoclonal antibody or binding protein may be readily determined by one of ordinary skill in the art (see Dower et al., “The Interaction of Monoclonal Antibodies with MHC Class I Antigens on Mouse Spleen Cells. I. Analysis of the Mechanism of Binding,” J. Immunol. 132:751, 1984). Briefly, increasing amounts of radiolabeled antibody or binding protein are exposed to IL-1R bearing cells. An antibody's affinity may be determined by taking the reciprocal of the antibody concentration at which one-half of the antibodies maximally bind (see Dower et al., supra). As will be evident to one of ordinary skill in the art, antibodies may be generated against either whole IL-1R, or portions of the IL-1R. Particularly preferred are antibodies developed against the soluble truncated form of the IL-1R. Additionally, within the context of the present invention monoclonal antibodies include F(ab′)₂ and Fab fragments which may be readily prepared by one of ordinary skill in the art.

Polyclonal antibodies may be readily generated by one of ordinary skill in the art from a variety of warm-blooded animals such as horses, cows, various fowl, rabbits, mice, or rats. Briefly, IL-1R is utilized to immunize the animal through intraperitoneal, intramuscular, intraocular, or subcutaneous injections. The immunogenicity of IL-1R may be increased through the use of an adjuvant such as Freund's complete or incomplete adjuvant. Following several booster immunizations, small samples of serum are collected and tested for reactivity to IL-1R by any of a number of methods, including among others, assays discussed below in the Examples such as an ELISA, ABC or modified ABC assays, or by a dot blot assay. Particularly preferred polyclonal antisera will give a signal on one of these assays that is at least three times greater than background. Once the titer of the animal has reached a plateau in terms of its reactivity to the IL-1R, larger quantities of polyclonal antisera may be readily obtained either by weekly bleedings, or by exsanguinating the animal.

Monoclonal antibodies may also be readily generated using conventional techniques (see U.S. Pat. Nos. RE 32,011, 4,902,614, 4,543,439, and 4,411,993 which are incorporated herein by reference; see also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference). Briefly, within one embodiment a subject animal such as a rat or mouse is injected with a form of IL-1R suitable for generating an immune response against the IL-1R. This may be accomplished by immunization with various forms of the IL-1R, including among others, cells which express the IL-1R, viruses such as the vaccinia virus which express the IL1R, soluble forms of the IL-1R, or peptides which are based upon the IL-1R sequence. Additionally, many techniques are known in the art for increasing the resultant immune response, for example by coupling the soluble receptor or peptide to another protein such as ovalbumin or keyhole limpet hemocyanin (KLH), or through the use of adjuvants such as Fruend's complete or incomplete adjuvant. The initial immunization may be through intraperitoneal, intramuscular, intraocular, or subcutaneous routes.

Between one and three weeks after the initial immunization the animal may be reimmunized with another booster immunization. The animal may then be test bled and the serum tested for immunoreactivity to the IL-1R using assays such as an ELISA, dot blot, ABC or modified ABC assay as described below in the Examples. Additional immunizations may also be accomplished until the animal has plateaued in its reactivity to the IL-1R. The animal may then be given a final boost of soluble IL-1R, and three to four days later sacrificed. At this time, organs which contain large numbers of B cells such as the spleen and lymph nodes may be harvested and disrupted into a single cell suspension by passing the organs through a mesh screen or by rupturing the spleen or lymph node membranes which encapsidate the cells. Within one embodiment the red cells are subsequently lysed by the addition of a hypotonic solution, followed by immediate return to isotonicity.

Within another embodiment, suitable cells for preparing monoclonal antibodies are obtained through the use of in vitro immunization techniques. Briefly, an animal is sacrificed and the spleen and lymph node cells are removed as described above. A single cell suspension is prepared, and the cells are placed into a culture which contains a form of the IL-1R, which is suitable for generating an immune response as described above. Subsequently, the lymphocytes are harvested and fused as described below.

Cells which are obtained through the use of in vitro immunization or from an immunized animal as described above may be immortalized by transfection with a virus such as the Epstein bar virus (EBV) (see Glasky and Reading, Hybridoma 8(4):377-389, 1989). Alternatively, within a preferred embodiment, the harvested spleen and/or lymph node cell suspensions are fused with a suitable myeloma cell in order to create a “hybridoma” which secretes monoclonal antibody. Suitable myeloma lines are preferably defective in the construction or expression of antibodies, and are additionally syngeneic with the cells from the immunized animal. Many such myeloma cell lines are well known in the art and may be obtained from sources such as the American Type Culture Collection (ATCC), Rockville, Md. (see Catalogue of Cell Lines & Hybridomas, 6th ed., ATCC, 1988). Representative myeloma lines include: for humans UC 729-6 (ATCC No. CRL 8061), MC/CAR-Z2 (ATCC No. CRL 8147), and SKO-007 (ATCC No. CRL 8033); for mice SP2/0-AG14 (ATCC No. CRL 1581), and P3X63Ag8 (ATCC No. TIB 9), and for rats Y3-Agl.2.3 (ATCC No. CRL 1631), and YB2/0 (ATCC No. CRL 1662). Particularly preferred fusion lines include NS-1 (ATCC No. TIB 18), and P3×63—Ag 8.653 (ATCC No. CRL 1580) which may be utilized for fusions with either mouse, rat, or human cell lines. Fusion between the myeloma cell line and the cells from the immunized animal may be accomplished by a variety of methods, including the use of polyethylene glycol (PEG) (see Antibodies: A Laboratory Manual, Harlow and Lane (eds.), Cold Spring Harbor Laboratory Press, 1988) or electrofusion (see Zimmerman and Vienken, J. Membrane Biol. 67:165-182, 1982).

Following the fusion, the cells may be placed into culture plates containing a suitable medium, such as RPMI 1640, or DMEM (Dulbecco's Modified Eagles Medium) (JRH Biosciences, Lenexa, Kans.). The medium may also contain additional ingredients, such as Fetal Bovine Serum (FBS, Le., from Hyclone, Logan, Utah, or JRH Biosciences), thymocytes which were harvested from a baby animal of the same species as was used for immunization, or agar to solidify the medium. Additionally, the medium should contain a reagent which selectively allows for the growth of fused spleen and myeloma cells. Particularly preferred is the use of HAT (hypoxanthine, aminopterin, and thymidine) (Sigma Chemical Co., St. Louis, Mo.). After about seven days, the resulting fused cells or hybridomas may be screened as described below in Example 15 in order to determine the presence of antibodies which recognize IL-1R. Following several clonal dilutions and reassays, a hybridoma producing antibodies which bind to IL-1R may be isolated.

Other techniques may also be utilized to construct monoclonal antibodies (see William D. Huse et al., “Generation of a Large Combinational Library of the Immunoglobulin Repertoire in Phage Lambda,” Science 246:1275-1281, December 1989; see also L Sastry et al., “Cloning of the Immunological Repertoire in Escherichia coli for Generation of Monoclonal Catalytic Antibodies: Construction of a Heavy Chain Variable Region-Specific cDNA Library,” Proc. Natl. Acad. Sci. USA 86:5728-5732, August 1989; see also Michelle Alting-Mees et al., “Monoclonal Antibody Expression Libraries: A Rapid Alternative to Hybridomas,” Strategies in Molecular Biology 3:1-9, January 1990; these references describe a commercial system available from Stratacyte, La, Jolla, Calif., which enables the production of antibodies through recombinant techniques). Briefly, mRNA is isolated from a B cell population, and utilized to create heavy and light chain immunoglobulin cDNA expression libraries in the μImmunoZap(H) and μImmunoZap(L) vectors. These vectors may be screened individually or co-expressed to form Fab fragments or antibodies (see Huse et al., supra; see also Sastry et al., supra). Positive plaques may subsequently be converted to a non-lytic plasmid which allows high level expression of monoclonal antibody fragments from E. coli.

Similarly, binding proteins may also be constructed utilizing recombinant DNA techniques to incorporate the variable regions of a gene which encodes a specifically binding antibody. The construction of these proteins may be readily accomplished by one of ordinary skill in the art (see James W. Larrick et al., “Polymerase Chain Reaction Using Mixed Primers: Cloning of Human Monoclonal Antibody Variable Region Genes From Single Hybridoma Cells,” Biotechnology 7:934-938, September 1989; Riechmann et al., “Reshaping Human Antibodies for Therapy,” Nature 332:323-327, 1988; Roberts et al., “Generation of an Antibody with Enhanced Affinity and Specificity for its Antigen by Protein Engineering,” Nature 328:731-734, 1987; Verhoeyen et al., “Reshaping Human Antibodies: Grafting an Antilysozyme Activity,” Science 239:1534-1536, 1988; Chaudhary et al., “A Recombinant Immunotoxin Consisting of Two Antibody Variable Domains Fused to Pseudomonas Exotoxin,” Nature 339:394-397, 1989), given the disclosure provided herein. Briefly, the antigen-binding sites or IL-1 receptor binding domain from a cell which produces a specifically binding monoclonal antibody are amplified, and inserted directly into the genome of a cell which produces human antibodies (see Verhoeyen et al., supra; see also Reichmann et al., supra). This technique allows the antigen-binding site of a specifically binding murine or rat monoclonal antibody to be transferred into a human antibody. Such antibodies are preferable for therapeutic use in humans because they are not as antigenic as rat or mouse antibodies. Alternatively, the antigen-binding sites (variable region) may be either linked to, or inserted into, another completely different protein (see Chaudhary et al., supra), resulting in a new protein with antigen-binding sites of the antibody as well as the functional activity of the completely different protein. As one of ordinary skill in the art will recognize, the antigen-binding sites or IL-1 receptor binding domain of the antibody may be found in the variable region of the antibody. Furthermore, DNA sequences which encode smaller portions of the antibody or variable regions which specifically bind to mammalian IL-1R may also be utilized within the context of the present invention. These portions may be readily tested for binding specificity to the IL-1R utilizing assays described below in Example 15, including for example ELISA, ABC, or dot blot assays.

Within a preferred embodiment, the genes which encode the variable region from a hybridoma producing a monoclonal antibody of interest are amplified using nucleotide primers for the variable region. These primers may be synthesized by one of ordinary skill in the art, or may be purchased from commercially available sources. Stratacyte (La Jolla, Calif.) sells primers for mouse and human variable regions including, among others, primers for V_{Ha}, V_{Hb}, V_{Hc}, V_{Hd}, C_{H1}, V_{L} and C_{L} regions. These primers may be utilized to amplify heavy or light chain variable regions, which may then be inserted into vectors such as ImmunoZAP™ H or ImmunoZAP™ L (Stratacyte), respectively. These vectors may then be introduced into E. coli for expression. Utilizing these techniques, large amounts of a single-chain protein containing a fusion of the V_{H} and V_{L} domains may be produced (see Bird et al., Science 242:423-426, 1988).

Within another embodiment, the binding protein is fused within the expression vector to another protein, such as a toxin. Cells which are bound by the binding protein may thus be killed by incorporation of the toxin (see Chaudhary et al.). Alternatively, the binding protein may be fused to an IL-1 antagonist (Le., a protein which binds IL-1 receptor but generates no biological activity), allowing large local concentrations of the antagonist to be developed around cells which express IL-1 receptor. Only cells which could bind the antagonist would be affected, potentially decreasing the dose needed for therapeutic purposes.

Once suitable antibodies or binding proteins have been obtained, they may be isolated or purified by many techniques well known to those of ordinary skill in the art (see Antibodies: A Laboratory Manual, Harlow and Lane (eds.), Cold Spring Harbor Laboratory Press, 1988). Suitable techniques include peptide or protein affinity columns, HPLC or RP-HPLC, purification on protein A or protein G columns, or any combination of these techniques.

Antibodies and binding proteins of the present invention have many uses. For example, antibodies may be utilized in flow cytometry to sort IL-1R bearing cells, or to histochemically stain IL-1R bearing cells. Briefly, in order to detect IL-1 receptors on cells, the cells are incubated with a labeled monoclonal antibody which specifically binds to mammalian IL-1 receptors, followed by detection of the presence of bound antibody. These steps may also be accomplished with additional steps such as washings to remove unbound antibody. Labels suitable for use within the present invention are well known in the art including, among others, Flourescein Isothiocyanate (FHTC), Phycoerythrin (PE), Horse Radish Peroxidase (HRP), and colloidal gold. Particularly preferred for use in flow cytometry is FITC which may be conjugated to purified antibody according to the method of Keltkamp in “Conjugation of Fluorescein Isothiocyanate to Antibodies. I. Experiments on the Conditions of Conjugation,” Immunology 18:865-873, 1970. (See also Keltkamp, “Conjugation of Fluorescein Isothiocyanate to Antibodies. II. A Reproducible Method,” Immunology 18:875-881, 1970; and Goding, “Conjugation of Antibodies with Fluorochromes: Modification to the Standard Methods,” J. Immunol. Methods 13:215-226, 1970.) For histochemical staining, HRP is preferred, which may be conjugated to the purified antibody according to the method of Nakane and Kawaoi in “Peroxidase-Labeled Antibody: A New Method of Conjugation,” J. Histochem. Cytochem. 22:1084-1091, 1974. (See also Tijssen and Kurstak, “Highly Efficient and Simple Methods for Preparation of Peroxidase and Active Peroxidase Antibody Conjugates for Enzyme Immunoassays,” Anal. Biochem 136:451-457, 1984.)

Purified antibodies or binding proteins may also be utilized therapeutically to block the binding of IL-1 to the receptor in vivo, or for in vivo neutralization of IL-1R bearing cells. Within preferred embodiments, the antibody is modified to escape immunological detection, for example, by transferring the antigen-binding site of a specific murine monoclonal antibody to a human monoclonal antibody, as discussed above. Particularly preferred is the use of therapeutic compositions comprising an antibody or binding protein to the IL-1 receptor, and a physiologically acceptable carrier or diluent. Suitable carriers or diluents include, among others, neutral buffered saline or saline mixed with nonspecific albumin. Additionally, the therapeutic composition may include further excipients or stabilizers such as buffers, carbohydrates including, for example, glucose, sucrose, or dextrose, chelating agents such as EDTA, or various preservatives. Appropriate dosages may be determined in clinical trials, although the amount and frequency of administration may be dependent on such factors as the nature and severity of the indication being treated, the desired response, and the condition of the patient.

Antibodies may also be utilized to monitor the presence of circulating soluble IL-1R which has been administered to a patient, or to measure in vivo levels of IL-1R in patients. Within a preferred embodiment, a double determinant or sandwich assay is utilized to detect the IL-1R. Briefly, serum suspected of containing soluble IL-1R is incubated with a solid support having a monoclonal antibody, as described above, affixed thereto under conditions and for a time sufficient for binding to occur. Many solid supports are known in the art, including, among others, ELISA plates (Linbro, McLean, Va.), nitrocellulose (Millipore Corp. Bedford, Mass.), beads (Polysciences, Warrington, Penn.), and magnetic beads (Robbin Scientific, Mountain View, Calif.). Additionally, the monoclonal antibody may be readily affixed to the solid support utilizing techniques well known in the art (see Antibodies: A Laboratory Manual, Harlow and Lane (eds.), Cold Spring Harbor Laboratory Press, 1988). The solid support is then incubated with a second labeled monoclonal antibody specific for mammalian IL-1 receptors under conditions and for a time sufficient for binding to occur, after which presence of bound labeled antibody may be detected.

Within a particularly preferred embodiment, a monoclonal antibody is coated onto a solid support such as a 96 well plate. Subsequently, the plate is blocked with a protein such as bovine serum albumin or nonfat dry milk for about 30 minutes. Serum from a patient is diluted in phosphate buffered saline and incubated in the wells under conditions and for a time sufficient for binding to occur—generally about 30 minutes. Subsequently, the plate is washed and a labeled second monoclonal antibody specific for a different IL-1R epitope is added into the wells and incubated as described above. Antibodies for different IL-1R may be determined through the use of cross-blocking assays, as described below in Example 15. The well is then examined for the presence of the second labeled antibody. Presence of the second labeled antibody indicates the presence of the IL-1R in the patient's serum. As will be understood by one of ordinary skill in the art, the monoclonal antibodies used within the above assay may be substituted with polyclonal antibodies or binding proteins which are specific for the IL-1 receptor.

The following examples are offered by way of illustration, and not by way of limitation.

### EXAMPLES

### Example 1

### Preparation of IL-1α Affinity matrix and Affinity Purification of Receptor From Surface Labeled EL-4 6.1 C10 Cells

Cell surface proteins on EL-4 6.1 C10 cells were radiolabeled with ¹²⁵I by the glucose oxidase-lactoperoxidase method disclosed by Cosman et al. (Molecular Immunol. 23:935, 1986). Labeled cells were pelleted by centrifugation, washed three times with PBS, and extracted with PBS containing 1% Triton X-100 and the cocktail of protease inhibitors described in the assay protocol detailed above. The Triton X-100 extract was spun for 10 minutes in an Eppendorf microcentrifuge and the supernatant was stored at −70° C.

Recombinant IL-1α was coupled to cyanogen bromide activated Sepharose 4B (Pharmacia, Piscataway, N.J., USA) or to Affigel-10 (Bio-Rad, Richmond, Calif., USA) according to the manufacturer's suggestions. For example, to a solution of IL-1α (1.64 mg/ml in 9.5 ml PBS), 3 ml were added of swollen, acid-washed, CNBr-activated Sepharose. The solution was rocked overnight at 4° C. and an aliquot of the supernatant was tested for protein by a fluorescamine protein assay as described by Udenfriend et al. (Science 178:871, 1972), using BSA as a standard. Ninety-eight percent of the protein had coupled to the gel, suggesting that the column had a final load of 5.1 mg IL-1α per ml gel. Three hundred μl of 1 M glycine-ethyl-ester (Sigma Chemical Co., St. Louis, Mo., USA) were added to the slurry to block any unreacted sites on the gel.

The gel was washed extensively with 0.1 M glycine buffer pH 3.0 containing 0.1% Triton X-100, PBS containing 0.1% Triton X-100, RIPA buffer (0.05 M Tris-HCl pH 7.5, 0.15 M NaCl, 1% NP40, 1% sodium deoxycholate, 0.1% SDS), and PBS containing 0.1% Triton X-100 and 10 mM ATP. Small columns (200 μl¹) were prepared in disposable polypropylene holders (Bio-Rad, Richmond, Calif., USA) and washed with PBS containing 1% Triton X-100. Aliquots of 100 μl of ¹²⁵I-labeled extract were applied to a column, which was then washed with PBS containing 1% Triton X-100, RIPA buffer, PBS containing 0.1% Triton X-100 and 10 mM ATP, and PBS with 1% Triton X-100.

The IL-1 receptor on murine T cells is a robust structure capable of binding ¹²⁵I-IL-1α in Triton X-100 detergent solutions. To be able to recover receptor from such an affinity matrix, a mild elution procedure is necessary. Mild acid treatment can cause rapid dissociation of preformed IL-1α/IL-1 receptor complexes. Based upon this observation, pH 3.0 glycine HCl buffer containing 0.1% Triton X-100 were used to elute receptor from the IL-1α affinity columns, which was collected in 0.05 ml fractions. The presence of receptor in the fractions was detected by dot blot as described above, using ¹²⁵I-labeled IL-1α.

Analysis by SDS-PAGE proceeded as follows. To 50 μl of each column fraction was added 50 μl of 2×SDS sample buffer (0.125 M Tris HCl pH 6.8, 4% SDS, 20% glycerol, 10% 2-mercaptoethanol). The solution was placed in a boiling water bath for three minutes and aliquots of 40 μl were applied to the sample well of a 10% polyacrylamide gel which was set up and run according to the method of Laemmli (Nature 227:680, 1970). Gels were fixed and stained using 0.25% Coomassie brilliant blue in 25% isopropanol, 10% acetic acid) destained in 25% isopropanol, 10% acetic acid, treated with Enhance (New England Nuclear, Boston, Mass., USA) dried and exposed to Kodak X-Omat AR film at −70° C. Molecular weight markers, labeled with ¹⁴C, were obtained from New England Nuclear, and included: cytochrome C (Mᵣ 12,300), lactoglobulin A (Mᵣ 18,367), carbonic anhydrase (Mᵣ 31,000,), ovalbumin (Mᵣ 46,000), bovine serum albumin (Mᵣ 69,000), phosphorylase B (Mᵣ 97,400) and myosin (Mᵣ 200,000). Alternatively, fractions having receptor activity were analyzed by SDS polyacrylamide gel electrophoresis followed by silver staining as previously described by Urdal et al. (Proc. Natl. Acad. Sci. USA 81:6481, 1984).

Dot blot analysis of fractions eluted from the IL-1α affinity matrix showed that IL-1 binding activity was detected in fractions that were collected after pH 3.0 glycine buffer was applied to the column. Fractions that scored positive in this assay, when analyzed by SDS-PAGE, revealed that a protein of Mᵣ 82,000 could be detected upon developing the gel with silver stain. To determine which of the proteins detected by silver stain were expressed on the cell surface, EL-4 6.1 cells were surface labeled with ¹²⁵I by the lactoperoxidase-glucose oxidase procedure. Radiolabeled cells were then extracted with PBS containing 1% Triton X-100 and aliquots of the detergent extract applied to an IL-1α affinity matrix. Fractions that were collected from this column, following application to the column of pH3.0 glycine buffer, contained a radiolabeled protein of Mᵣ 82,000.

### Example 2

### Comparison of Properties of Cellular IL-1 Receptor and IL-1 Receptor Isolated From Cell Extracts

In a preliminary experiment, the binding properties of the IL-1 receptor were compared in intact EL-4 6.1 C10 cells and after extraction from cells. 3.8×10⁸ EL-4 6.1 C10 cells were divided into two equal aliquots, one of which was extracted as described above. The remaining cells were resuspended at 3.8×10⁷ cells/ml and used for direct binding studies. Extract was adsorbed to nitrocellulose and used for solid phase binding studies employing various concentrations of ¹²⁵I-IL-1α with or without unlabeled IL-1. After washing and drying, the nitrocellulose filters were first counted for bound ¹²⁵I-IL-1α and subsequently placed on film for autoradiography. Nonspecific background was measured in the presence of 5.7×10⁻⁷ M unlabeled rIL-1 β. The data obtained showed that ¹²⁵I-IL-1α was bound to the extract on nitrocellulose in an IL-1 concentration-dependent fashion, and that the ¹²⁵I-IL-1α was specifically bound to the region of the blot where extract is present. Further, binding could be extensively blocked by inclusion of unlabeled IL-1α in the incubation mixture.

The comparison further indicated that not only were the levels of receptor the same in both instances, but that the receptors after adsorption to nitrocellulose exhibited an affinity for ligand which was indistinguishable from that of the receptor in intact cells. No significant difference between the number of receptors detected on intact cells and those detected following detergent extraction was found. This is consistent with the view that the majority of the receptors were present on the external face of the plasma membrane in intact cells.

To measure the specificity of binding of IL-1 receptors on nitrocellulose filters, 2 μl of EL-4 6.1 C10 extract were applied to nitrocellulose filters, dried, blocked and assayed as described above. The following proteins were tested for their capacity to inhibit ¹²⁵I-IL-1 binding: human rIL-1α(7.62×10⁻⁷ M), human rIL-1 (7.62×10-7 M), human IL-2 (8.9×10-7 M), murine IL-3 (7.5×10⁻⁴ M), murine-GM-CSF (7.5×10⁻⁷ M), recombinant murine IL-4 (5×10⁻⁹ M), human epidermal growth factor 3 μg/ml, fibroblast growth factor 1 μg/ml, rat submandibular gland nerve growth factor (2 μg/ml), bovine insulin (1×10⁷ M), human luteinizing hormone 1 μg/ml), human growth hormone 1.7×10⁻⁷), thyroid stimulating hormone (1 μg/ml), and follicle stimulating hormone (1 μg/ml). All incubations were done with 1.9×10⁻¹⁰ M ¹²⁵I-IL-1α.

This experiment demonstrated that extracted receptor retains the same specificity as that previously demonstrated for intact cells. As found with intact cells, only IL-1α and IL-1: produced any significant inhibition of ¹²⁵I-IL-1α binding. The data showed that unlabeled IL-1α and IL-1β produced >90% inhibition of ¹²⁵I-IL-1α binding, while no significant blockade was observed with any of the other hormones.

To determine whether receptor in detergent solution would bind IL1 with an affinity equal to that of receptor in cell membranes, or adsorbed to nitrocellulose, a third experiment was performed in which the nitrocellulose dot blot binding assay was used to test the capacity of an EL-4 6.1 C10 extract in Triton X-100 solution to inhibit binding of ¹²⁵I-IL-1α to the solid phase. EL-4 6.1 C10 extracts were adsorbed to nitrocellulose, dried, blocked and incubated with mixture of ¹²⁵I-IL-1α and extracts containing receptors in detergent solution.

The concentration of receptor in the solution phase was estimated from a saturation binding curve to 1 μl aliquots blotted on nitrocellulose, allowing receptors/μl to be calculated and hence IL-1 receptor concentration (M). The extract was diluted through PBS Triton X-100 solution (0.5% Triton) to keep the detergent concentration constant. The inhibition curve showed that in solution, the receptor bound to ¹²⁵I-IL-1α with a Kₐ (4.5±0.5×10⁹ M⁻¹) that is the same as that of receptor on the solid phase or in membranes. Further, the close fit between the theoretical curve, which is based on a simple competitive inhibition model, and the data was consistent with the hypothesis that a single type of IL-1 binding protein was present in the membrane extract.

In order to examine the integrity of the receptor as a function of the concentration of total EL-4 6.1 C10 extract membrane proteins, a fourth experiment was done. Mixtures of EL-4 6.1 C10 extract in various proportions ranging from 10% to 100% were made either with an extract from cells not expressing the IL-1 receptor, EL-4 (M) cells, or with PBS Triton X-100 (0.5%). Each mixture was analyzed for receptor concentration, and affinity of ¹²⁵I-IL-1α binding by quantitative dot blot binding. Receptor concentration decreased linearly with the percentage of EL-4 6.1 C10 extract present, whether membrane protein concentration was maintained at a constant level or not. In both series of mixtures the affinity of the receptor for ¹²⁵I-IL-1α remained constant. These data are consistent with one of two hypotheses, either the receptor binding function is contained within a single polypeptide chain or, if the functional receptor requires two or more subunits for IL-1 binding, these are sufficiently tightly associated that dilution through detergent does not separate them.

### Example 3

### Purification of IL-1 Receptor to Homogeneity and Determination of N-Terminal Sequence

300-500 liters of EL-4 6.1 C10 cells were grown to saturation under the conditions previously described, harvested, and extracted with PBS-1% Triton X-100. The detergent extract was applied to an IL-1α affinity column and the column washed as previously described. Fractions containing IL-1 receptor were detected by the ¹²⁵I-IL1α dot blot procedure following elution of the column with 0.1 M glycine HCl pH 3.0 containing 0.1% Triton X-100. Aliquots of the fractions were analyzed by SDS polyacrylamide gel electrophoresis.

This partially purified IL-1 receptor composition prepared by affinity chromatography on Affigel-IL1α was adjusted to contain the following buffer composition: 10 mM Tris-HCl, pH18, 250 mM NaCl, 0.5 mM MgCl₂, 0.5 mM MnCl₂, 0.5 mM CaCl₂, and 0.01% (v/v) Triton X-100 (WGA buffer). The IL-1 receptor composition was then applied to a 1 ml column of wheat germ agglutinin (WGA) bound to Sepharose CL-6B, equilibrated with WGA buffer. Following application of the IL-1 receptor composition, the WGA column was washed with 20 ml of WGA buffer followed by 10 mM Tris HCl, pH 8, 0.01% (v/v) Triton X-100. The IL-1 receptor protein was eluted from the WGA column with 10 mM Tris-HCl, pH 8, 0.5 M N-acetylglucosamine, and 0.01% (v/v) Triton X-100. The presence of biologically active IL-1 receptor was detected by the ¹²⁵IL-1α dot blot procedure. The fractions were also analyzed by SDS polyacrylamide gel electrophoresis followed by silver staining.

Material eluting from the WGA column was applied to a C8 RP-HPLC column. The C8 RP-HPLC column (Brownlee Labs RP-300, 1 mm×50 mm) was previously equilibrated with 0.1% (v/v) trifluoroacetic acid (TFA) in HPLC grade H₂O, at a flow rate of 50 μl/min. Following application of the IL-1 receptor containing material, the C8 RP-HPLC column was washed with 0.1% (v/v) TFA in H₂O at 50 μl/min until the absorbance at 280 nm returned to baseline. The IL-1 receptor protein was eluted from the column by running a linear gradient of 0.1% (v/v) TFA in acetonitrile from 0-100% at a rate of 1% per minute. Aliquots of the fractions were analyzed by SDS polyacrylamide gel electrophoresis. The IL-1 receptor protein was found to consist of a single band on an SDS polyacrylamide gel migrating with a molecular weight of 82,000.

The purified IL-1 receptor protein was analyzed by Edman degradation using an Applied Biosystems Model 470A protein sequencer. The protein (150 picomoles) was not modified before analysis. The results of the N-terminal protein sequence analysis of the IL-1 receptor indicated the following sequence of amino acid residues: NH₂-Leu-Glu-Ile-Asp-Val-Cys-Thr-Glu-TyrPro-Asn-Gln-Ile-Val-Leu-Phe-Leu-Ser-Val-Asn-Glu-Ile-Asp-Ile-Arg-Lys.

This protein sequence was found to be unique when compared to the Mar. 17, 1987 release of the Protein Sequence Database of the Protein Identification Resource of the National Biomedical Research Foundation. This release of the database contained 4,253 sequences consisting of 1,029.056 residues.

### Example 4

### Isolation of cDNA Encoding Murine IL-1R by Direct Expression of Active Protein in COS-7 Cells

A cDNA library was constructed by reverse transcription of polyadenylated mRNA isolated from total RNA extracted from EL-4 6.1 C10 cells by a procedure similar to that of Chirgwin et al. (Biochem. 18:5294, 1979). Briefly, the cells were lysed in a guanidinium isothiocyanate solution, and the lysate layered over a pad of CsCl and centrifuged until the RNA had pelleted. The RNA pellet was resuspended and further purified by protease digestion, organic extraction and alcohol precipitation. Poly A⁺ RNA was isolated by oligo dT cellulose chromatography and double-stranded cDNA was prepared by a method similar to that of Gubler and Hoffman (Gene 25:263, 1983). Briefly, the RNA was copied into cDNA by reverse transcriptase using either oligo dT or random oligonucleotides as primer. The cDNA was made double-stranded by incubation with E. coli DNA polymerase I and RNase H, and the ends made flush by further incubation with T₄ DNA polymerase. The blunt-ended cDNA was ligated into SmaI-cut dephosphorylated pDC201 vector DNA.

The eukaryotic high expression vector pDC201 was assembled from SV40, adenovirus 2, and pBR322 DNA comprising, in sequence: (1) an SV40 fragment containing the origin of replication, early and late promoters, and enhancer; (2) an adenovirus 2 fragment containing the major late promoter, the first exon and part of the first intron of the tripartite late leader; (3) a synthetic sequence comprising a HindIII site, a splice acceptor site, the second and third exons of the adenovirus 2 tripartite leader and a multiple cloning site including a SmaI site: (4) additional SV40 sequences containing early and late polyadenylation sites; (5) adenovirus 2 sequences including the virus-associated RNA genes; and (6) pBR322 elements for replication in E. coli.

The resulting EL-4 6.1 C10 cDNA library in pDC201 was used to transform E. coli strain DH5α, and recombinants were plated to provide approximately 350 colonies per plate and sufficient plates to provide approximately 25,000 total colonies per screen. Colonies were scraped from each plate, pooled, and plasmid DNA prepared from each pool. The pooled DNA was then used to transfect a sub-confluent layer of monkey COS-7 cells using DEAE-dextran followed by chloroquine treatment, as described by Luthman et al. (Nucleic Acids Res. 11:1295, 1983) and McCutchan et al. (J. Natl. Cancer Inst. 41:351, 1986). The cells were then grown in culture for three days to permit transient expression of the inserted sequences. After three days, cell culture supernatants were discarded and the cell monolayers in each plate assayed for IL1 binding as follows. Three ml of RPMI medium continuing 3×10⁻¹⁰M ¹²⁵I-IL-1α was added to each plate and the plates incubated for 2 hours at 80° C. This medium was then discarded, and each plate was washed with 10 ml RPMI 1640 medium (containing no labeled IL-1α). The edges of each plate were then broken off, leaving a flat disk which was contacted with X-ray film for 72 hours at −70° C. using an intensifying screen. IL-1 binding activity was visualized on the exposed films as a dark focus against a relatively uniform background.

After approximately 150,000 recombinants from the library had been screened in this manner, one transfectant pool was observed to provide IL-1 binding foci which were clearly apparent against the background exposure.

A frozen stock of bacteria from the positive pool was then used to obtain plates of approximately 350 colonies. Replicas of these plates were made on nitrocellulose filters, and the plates were then scraped and plasmid DNA prepared and transfected as described above to identify a positive plate. Bacteria from individual colonies from the nitrocellulose replicas of this plate were grown in 2 ml cultures, which were used to obtain plasmid DNA, which was transfected into COS-7 cells as described above. In this manner, a single clone, clone 78, was isolated which was capable of inducing expression of IL-1R in COS cells. The insert was subcloned into a plasmid derived from pBR322 (GEMBL) and sequenced by conventional techniques. The sequence is set forth in FIG. 2.

### Example 5

### Isolation of Human cDNA Clones Which Hybridize to Murine IL-1 Receptor Probe DNAs

A cDNA polynucleotide probe was prepared from the 2,356 base pair (bp) fragment of clone 78 (see Example 4) by nick-translation using DNA polymerase I. The method employed was substantially similar to that disclosed by Maniatis et al. (supra, p. 109).

A cDNA library was constructed by reverse transcription of polyadenylated mRNA isolated from total RNA extracted from the cultured cells of a human T-cell line designated clone 22, described by Acres et al. (J. Immunol. 138:2132, 1987). These cells were cultured in RPMI 1640 medium plus 10% fetal bovine serum as described by Acres et al. (supra), in the presence of 10 ng/ml OKT3 antibody and 10 ng/ml human IL-2. The cDNA was rendered double-stranded using DNA polymerase I, blunt-ended with T4 DNA polymerase, methylated with EcoRI methylase to protect EcoRI cleavage sites within the cDNA, and ligated to EcoRI linkers. The resulting constructs were digested with EcoRI to remove all but one copy of the linkers at each end of the cDNA, and ligated to EcoRI-cut and dephosphorylated arms of bacteriophage λgt10 (Huynh et al., DNA Cloning: A Practical Approach, Glover (ed.), IRL Press, pp. 49-78). The ligated DNA was packaged into phage particles using a commercially available kit (Stratagene Cloning Systems, San Diego, Calif., USA 92121) to generate a library of recombinants. Recombinants were plated on E. coli strain C600(HF1⁻) and screened by standard plaque hybridization techniques under conditions of moderate stringency (50° C., 6×SCC).

Following several rounds of screening, nine clones were isolated from the library which hybridized to the cDNA probe. The clones were plaque purified and used to prepare bacteriophage DNA which was digested with EcoRI. The digests were electrophoresed on an agarose gel, blotted onto nylon filters, and retested for hybridization. The clones were digested with EcoRI followed by preparative agarose gel electrophoresis, then subcloned into an EcoRI-cut derivative (pGEMBL) of the standard cloning vector pBR322 containing a polylinker having a unique EcoRI site, a BamHI site and numerous other unique restriction sites. An exemplary vector of this type is described by Dente et al. (Nucleic Acids Research 11:1645, 1983).

Restriction mapping and sequencing of a 4.8 kb human IL-1R clone indicated that the clone included a sequence encoding 518 amino acids which exhibited 80% amino acid sequence identity to the corresponding murine sequence in the extracellular, or N-terminal region distal to the transmembrane region, 63% identity in the transmembrane region, and 87% identity in the cytoplasmic, or C-terminal region. In addition, several cysteine residues and most N-linked glycosylation sites between the mouse and human sequences were conserved. A 440 bp EcoRI-NsiI fragment derived from the 5′ portion of the human IL-1R clone was ³²P-labeled by nick-translation as described above and used to screen a cDNA library produced by randomly-priming clone 22 mRNA prepared as described above. Twenty-three clones which hybridized to the probe were isolated and analyzed by restriction mapping. Sequencing of one of these clones provided the sequence information corresponding to the remaining N-terminal 34 amino acids of the human protein. The coding and deduced amino acid sequence of the complete coding region of human IL-1R is shown in FIGS. 5A-5C.

### Example 6

### Expression of Recombinant IL-1 Receptor Using A High-Efficiency Mammalian Expression System

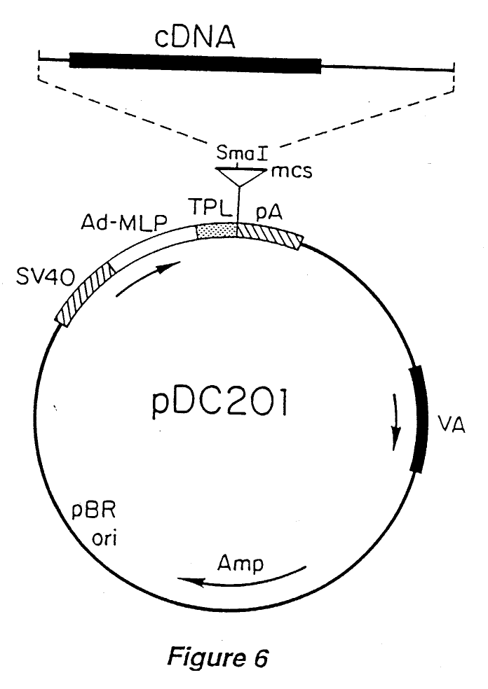

The mammalian expression plasmid pDC201, depicted in FIG. 6, is designed to express cDNA sequences inserted at its multiple cloning site (MCS) when transfected into mammalian cells. Referring now to FIG. 6, pDC201 includes the following components: SV40 (hatched box) contains SV40 sequences from coordinates 5171-270 including the origin of replication, enhancer sequences and early and late promoters. The fragment is oriented so that the direction of transcription from the early promoter is as shown by the arrow. Ad-MLP (open box) contains adenovirus-2 sequences from coordinates 5779-6231 including the major late promoter, the first exon and part of the intron between the first and second exons of the tripartite leader. TPL (stippled box) contains a synthetic DNA sequence specifying adenovirus-2 sequences 7056-7172, 9634-9693 (containing the acceptor splice site of the second exon of the tripartite leader, the second exon and part of the third exon of the tripartite leader) and a multiple cloning site (MCS) containing sites for KpnI, SmaI, and BgII. pA (hatched box) contains SV40 sequences from 4127-4100 and 2770-2533 that include the polyadenylation and termination signals for early transcription. VA (solid box) contains adenovirus-2 sequences from 10226-11555 that include the virus-associated RNA genes (VAI and VAII). The solid lines are derived from pBR 322 and represent (starting after the pA sequences and proceeding clockwise) coordinates 29-23, 651-185 (at which point the VA sequences are inserted), 29-1, 4363-2486, and 1094-375. pDC201 is a derivative of pMLSV, previously described by Cosman et al., Molec. Immunol. 23:935, 1986.

To express recombinant IL-1 receptor, COS cells were grown and transfected as described by Cosman et al., supra, with the plasmid DNA from a 1.5 ml culture of E. coli transformed with pDC201 having an IL-1R cDNA insert (clone 78). After 72 hours of culture cells were harvested by washing once with 10 ml of PBS and then treating for 20 minutes at 37° C. with an EDTA solution (sodium phosphate 0.05 M, sodium chloride 0.15 M, EDTA 0.005 M, pH 7.4) followed by scraping. For comparisons, COS cells were transfected with a pDC201 control vector containing no insert, and EL-4 6.1 C10 cells and EL-4 M cells (an IL-1 receptor-negative variant of EL-4 cells) were grown and harvested as described by McDonald et al., J. Immunol. 135:3964, 1985.

At saturating DNA concentrations, the transfected COS cell monolayer contained an average of 45,000 sites per cell. Since the parental COS cells expressed only about 500 receptors per cell, it can be calculated that more than 98% of all IL-1 receptors in the transfected population were recombinant. Flow cytometry using FITC-IL-1α revealed that only 4.2% of the cells stained brightly; therefore, each of these transfected COS cells contained about 1.1×10⁶ IL-1 binding sites.

The plasma membrane proteins of EL-4 6.1 C10 cells and of COS cells transfected with vector DNA containing cDNA encoding the IL-1 receptor (clone 78) were labeled with ¹²⁵I as described in Example 1, above. Cells were subsequently extracted with PBS containing 1% Triton X-100 and a cocktail of protease inhibitors (2 mM phenylmethyl sulphonyl fluoride, 1 mM pepstatin, 1 mM leupeptin, and 2 mM O-phenanthroline). Detergent extracts were subjected to affinity chromatography as described in Example 1 on Affigel-10 (Biorad, Richmond, Calif.) to which recombinant human IL-1α had been coupled. ¹²⁵I-labeled receptor was then eluted with sample buffer (0.0625 M Tris-HCl pH 6.8, 2% SDS, 10% glycerol, 5% 2-mercaptoethanol) and analyzed by SDS polyacrylamide gel electrophoresis on a 10% gel. Gels were then subjected to autoradiography. The recombinant IL-1 receptor purified by affinity chromatography in IL-1α columns migrated with a relative mobility of about 80,000 on SDS polyacrylamide gels, comparable to the mobility displayed by IL-1 receptor purified in the same manner from EL-4 6.1 C10 cells.

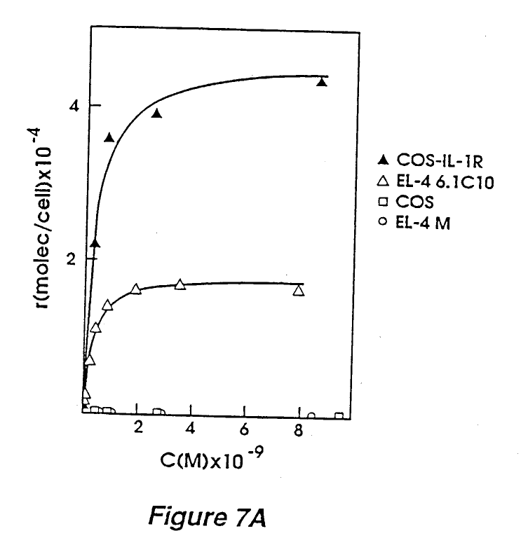

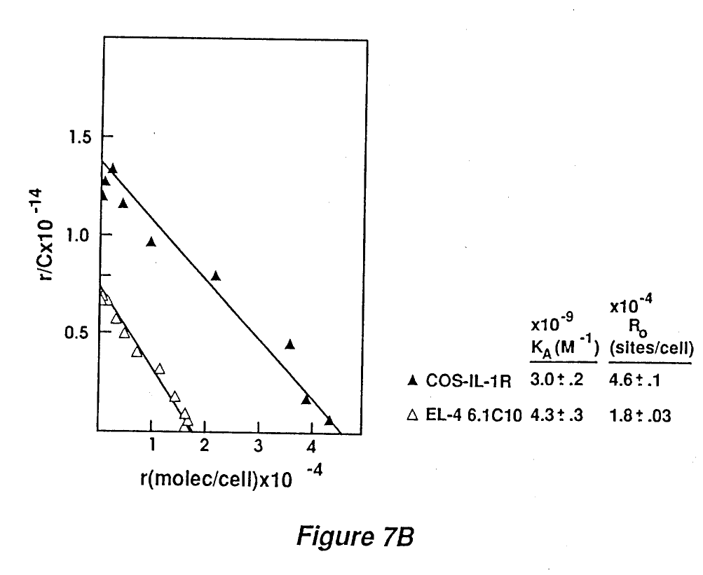

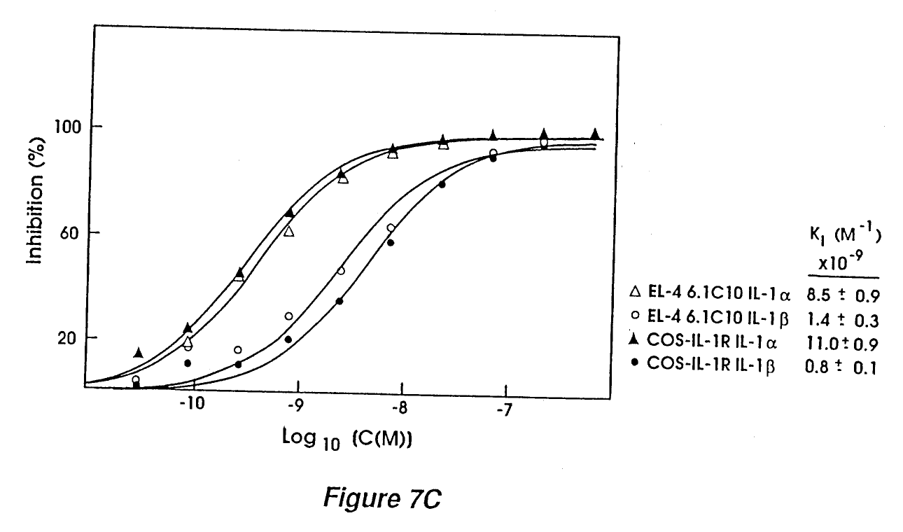

The DNA from clone 78, when transfected into COS cells, led to expression of IL-1 binding activity which was virtually identical to that displayed by EL-4 6.1 C10 cells, as shown in FIGS. 7A-7C.

For binding assays, COS cells were resuspended at 1.7×10⁶ cells/ml with EL-4 M (1.5×10⁷ cells/ml). All cell suspensions were made and binding assays done in RPMI 1640/10% BSA/0.1% sodium azide/20 mM HEPES pH 7.4. Binding incubations with ¹²⁵I-IL-1α or ¹²⁵I-IL-1β and unlabeled IL-1α and IL-1β were done as described elsewhere in the specification. ¹²⁵I-IL-1α bound to the transfected COS cells with a Kₐ of 3.0±0.2×10⁹ M⁻¹ (FIG. 7B). The Kₐ for the native receptor on EL-4 6.1 C10 cells was 4.3±3×10⁹ M⁻¹. All of the binding was to recombinant receptors (see FIG. 7A); the parental COS cell population did not bind detectable ¹²⁵I-IL-1α in this experiment.

In a cold competition experiment, free ¹²⁵I-IL-1α concentration was 7.72±0.13 10⁻¹⁰ M. On the transfected COS cells the maximal binding was 2.98±0.3×10⁴ molecules/cell (no inhibition) and the background (measured in the presence of 6×10⁻7 M unlabeled IL-1α) was 921±60 molecules/cell (100% inhibition). On the EL-4 6.1 C10 cells maximal binding was 1.33±0.02×10⁴ molecules/cell and background (see above) was 47±2 molecules/cell. Binding of ¹²⁵I-IL-1α, both to the transfected COS cells and to ELA 6.1 C10 cells, could be competed completely by an excess of either unlabeled IL-1α or unlabeled IL-1β (FIG. 7C). The inhibition constants for IL-1α and for IL-1β were very similar with each cell type (FIG. 7C).

### Example 7

### Preparation of Monoclonal Antibodies to IL-1R

Preparations of purified recombinant IL-1R, for example, human IL-1R, or transfected COS cells expressing high levels of IL-1R are employed to generate monoclonal antibodies against IL-1R using conventional techniques, for example, those disclosed in the U.S. Pat. No. 4,411,993. Such antibodies are likely to be useful in interfering with IL-1 binding to IL-1 receptors, for example, in ameliorating toxic or other undesired effects of IL-1.

To immunize mice, IL-1R immunogen is emulsified in complete Freund's adjuvant an injected in amounts ranging from 10-100 μg subcutaneously into Balb/c mice. Ten to twelve days later, the immunized animals are boosted with additional immunogen emulsified in incomplete Freund's adjuvant and periodically boosted thereafter on a weekly to biweekly immunization schedule. Serum samples are periodically taken by retro-orbital bleeding or tail-tip excision for testing by dot-blot assay, ELISA (enzyme-linked immunosorbent assay), or inhibition of binding of ¹²⁵I-IL-1α to extracts of EL-4 6.1 C10 cells (as described above). Other assay procedures are also suitable. Following detection of an appropriate antibody titer, positive animals are given an intravenous injection of antigen in saline. Three to four days later, the animals are sacrificed, splenocytes harvested, and fused to the murine myeloma cell line NS1. Hybridoma cell lines generated by this procedure are plated in multiple microtiter plates in a HAT selective medium (hypoxanthine, aminopterin, and thymidine) to inhibit proliferation of non-fused cells, myleoma hybrids, and spleen cell hybrids.

Hybridoma clones thus generated can be screened by ELISA for reactivity with IL-1R, for example, by adaptations of the techniques disclosed by Engvall et al., Immunochemistry 8:871, 1971, and in U.S. Pat. No. 4,703,004. Positive clones are then injected into the peritoneal cavities of syngeneic Balb/c mice to produce ascites containing high concentrations (>1 mg/ml) of anti-IL-1R monoclonal antibody. The resulting monoclonal antibody can be purified by ammonium sulfate precipitation followed by gel exclusion chromatography, and/or affinity chromatography based on binding of antibody to Protein A of Staphylococcus aureus.

### Example 8

### Expression OF IL-1R in Yeast

For expression of human or murine IL-1R in yeast, a yeast expression vector derived from pIXY120 is constructed as follows. pIXY120 is identical to pYaHuGM (ATCC 53157), except that it contains no cDNA insert and includes a polylinker/multiple cloning site with an NcoI site. This vector includes DNA sequences from the following sources: (1) a large SphI (nucleotide 562) to EcoRI (nucleotide 4361) fragment excised from plasmid pBR322 (ATCC 37017), including the origin of replication and the ampicillin resistance marker for selection in E. coli; (2) S. cerevisiae DNA including the TRP-1 marker, 2, origin of replication, ADH2 promoter; and (3) DNA encoding an 85 amino acid signal peptide derived from the gene encoding the secreted peptide α-factor (see Kurjan et al., U.S. Pat. No. 4,546,082). An Asp718 restriction site was introduced at position 237 in the α-factor signal peptide to facilitate fusion to heterologous genes. This was achieved by changing the thymine residue at nucleotide 241 to a cytosine residue by oligonucleotide-directed in vitro mutagenesis as described by Craik, Biotechniques 3(1):12-19,1985. A synthetic oligonucleotide containing multiple cloning sites and having the following sequence was inserted from the Asp718 site at amino acid 79 near the 3′ end of the α-factor signal peptide to a SpeI site in the 2, sequence:

```
Asp718 StuI NcoI BamHI
GTACCTTTGGATAAAAGAGACTACAAGGACGACGATGACAAGAGGCCTCCATGGAT. . .
        GAAACCTATTTTCTCTGATGTTCCTGCTGCTACTGTTCTCCGGAGGTACCTA. . .
                                                                                  |<----Polylinker--
          SmaI SpeI
...CCCCCGGGACA
...GGGGGCCCTGTGATC
---Polylinker--->|
```

pBC120 also varies from pYαHuGM by the presence of a 514 bp DNA fragment derived from the single-stranded phage f1 containing the origin of replication and intergenic region, which has been inserted at the Nru1 site in the pBR322 sequence. The presence of an f1 origin of replication permits generation of single-stranded DNA copies of the vector when transformed into appropriate strains of E. coli and superinfected with bacteriophage f1, which facilitates DNA sequencing of the vector and provides a basis for in vitro mutagenesis. To insert a cDNA, pIXY120 is digested with Asp718 which cleaves near the 3′ end of the α-factor leader peptide (nucleotide 237) and, for example, NcoI which cleaves in the polylinker. The large vector fragment is then purified and ligated to a DNA fragment encoding the protein to be expressed.

To create a secretion vector for expressing human IL-1R, a cDNA fragment including the complete open reading frame encoding hIL-1R is cleaved with an appropriate restriction endonuclease proximal to the N-terminus of, the mature protein. An oligonucleotide or oligonucleotides are then synthesized which are capable of ligation to the 5′ and 3′ ends of the hIL-1R fragment, regenerating any codons deleted in isolating the fragment, and also providing cohesive termini for ligation to pIXY120 to provide a coding sequence located in frame with respect to an intact α-factor leader sequence.

The resulting expression vectors are then purified and employed to transform a diploid yeast strain of S. cerevisiae (XV2181) by standard techniques, such as those disclosed in EPA 0165654, selecting for tryptophan prototrophs. The resulting transformants are cultured for expression of an hIL-1R protein as a secreted or extracted product. Cultures to be assayed for hIL-1R expression are grown in 20-50 ml of YPD medium (1% yeast extract, 2% peptone, 1% glucose) at 37° C. to a cell density of 1-5×10⁸ cells/ml. To separate cells from medium, cells are removed by centrifugation and the medium filtered through a 0.45 μ cellulose acetate filter prior to assay. Supernatants produced by the transformed yeast strain, or extracts prepared from disrupted yeasts cells, are assayed for the presence of hIL-1R using binding assays as described above.

### Example 9

### Construction, Expression and Purification of Truncated Recombinant Murine IL-1 Receptor

A truncated version of the IL-1 receptor protein was produced using an expression system compatible with the HELA-EBNA1 cell line, which constitutively expresses Epstein-Barr virus nuclear antigen driven from the CMV immediate-early enhancer promoter. The expression vector used was termed HAV-EO, a derivative of pDC201 which contains the Epstein-Barr virus origin and allows high level expression in the HELA-EBNA cell line. HAV-EO is derived from pDC201 by replacement of the adenovirus major late promoter with synthetic sequences from HIV-1 extending from the cap site of the viral mRNA, using the SV-40 early promoter to drive expression of the HIV-1 tat gene.

The expression construct for the soluble truncated IL-1 receptor was generated in a series of steps. The entire coding region of the receptor and part of the 5′ untranslated region were removed from the original IL-1 receptor clone 78 by digestion with Asp718 and NdeI. This fragment, containing no 3′ untranslated sequences, was cloned into HAV-EO, to generate HAV-EO-FL9. A variant of this plasmid, containing a translational stop codon immediately following the codon for proline 316 and lacking all the coding sequence 3′ to this, was subsequently constructed by standard methods and termed HAV-EO-MEXT.

HAV-EO-MEXT vector DNA was introduced into HELA-EBNA cells by a modified polybrene transfection as disclosed by Kawai and Nishizawa (Mol. Cell Biol. 4:1172, 1984). 1.5×10⁶ cells were seeded into 10 ml DMEM+10% FCS, in a 10 cm tissue culture dish. Cells were incubated at 37° C., 10% CO₂ for 16 hours. The media was then removed and 3 ml of serum-free DMEM containing 10 μg/ml DNA and 30 μg/ml polybrene (Sigma) were added. Dishes were then incubated at 37° C./10% CO₂ for a further six hours, at which time the DNA mix was removed and cells were glycerol shocked by addition of 3 ml serum-free DMEM+25% glycerol (v/v) for one minute. Glycerol was removed, and the cells were washed twice with medium. Ten ml of DMEM+10% FCs were then added, and the cells were incubated at 37°/10% CO₂ for 18 hours.

Transfected cells were then removed with trypsin and split in a ration of 1:9 into T175 cm² flasks (to give approximately 10% confluence) containing 25 ml DMEM+1% FCS. Supernatants containing transiently expressed soluble murine IL-1 receptor were harvested every 24 hours for up to ten days.

IL-1α binding activity in the medium was measured by inhibition of ¹²⁵I-1α to EL-4 6.1 C10 cells as described by Mosley et al. (J. Biol. Chem. 262:2941, 1987) with the exception that labeled IL-1α (2×10⁻¹¹, 50 μl) was first incubated with the test sample (50 μl) for two hours at 80° C., prior to addition of cells (2.5×10⁶ cells, 50 μl). Each test sample was assayed at six dilutions (X3) and the inhibition dose response curve used to assess the relative inhibitory titer.

Soluble IL-1 receptor was purified from culture supernatants as described for natural receptor by Urdal et al. (J. Biol. Chem. 263:280, 1988). Culture supernatants were passed over a 1 ml bed volume IL-1α column, the column was washed with PBS and eluted with 0.1 M glycine-HCl. Acid eluate fractions were immediately neutralized and subsequently tested for IL-1 binding activity using the radioreceptor inhibition assay. SDS-polyacrylamide gel electrophoresis of the material eluted by the acid treatment showed that it contained two bands of Mᵣ 60,000 and 54,000. N-glycanase treatment of this material indicated that the size heterogeneity is due to differences in N-linked glycosylation between the two species. Soluble IL-1 receptor retains full IL-1 bonding activity.

### Example 10

### Generation of Monoclonal Antibodies to Murine I-1R by Immunization With IL-1R Bearing C127 Cells

Full-length murine IL-1R was prepared and inserted into C127 cells (ATTC No. CRL 1616) as described by Dower et al. in J. Immun. 142(12):4314-4320, 1989. Briefly, pDC201 containing the full-length IL-1R cDNA was modified by addition of the entire bovine papilloma virus genome linearized at the BamHI site. The plasmid (BX8) was then transfected into C127 cells along with the plasmid PSV2 Neo at a ratio of 10:1, and cells expressing the IL-1R were selected.

A Lewis rat was immunized intraperitoneally with 10⁶ transformed C127 cells bearing recombinant murine IL-1R three times at three-week intervals. After the animal displayed antibody titer in an inhibition assay (as described below), it was boosted intravenously with two million whole C127 cells, and three days later sacrificed.

Spleen cells were harvested from the rat and fused with NS-1 (ATCC No. T1B 18) mouse myeloma cells at a 4:1 ratio with 50% PEG MW 1500 (#807489, EM Reagents, Schuchardt, West Germany) using standard fusion procedures. Six 96 well plates were seeded with cells from each fusion at a density of 2×10⁵ total cells per well in a volume of 200 μl. Plates were fed with 100 μl of HAT media on days seven and ten and were screened on day thirteen utilizing the ELISA method, as described below in Example 15. Three clones were selected on this basis for further analysis: mIL1Rm1, m3, and m5.

### Example 11

### Generation of Monoclonal Antibodies to Murine IL-1R BY Immunization With Soluble IL-1R

A Lewis rat was immunized subcutaneously with 30 μg of purified soluble IL-1R in complete Fruend's adjuvant, followed by booster immunizations twice a week with purified soluble IL-1R in incomplete Fruend's adjuvant. After several immunizations, a sera titer of 1:1,600 (as determined by an ABC assay) had developed. A final IV boost with 5 μg of soluble IL-1R was given prior to fusion. The fusion was accomplished essentially as described above in Example 10 utilizing P3X63 Ag8.653 (ATCC No. CRL 1580) as the myeloma cell line. Fusion plates were screened by the ABC method as described below, and 5 positives were selected on this basis: mIL1Rm15, m16, m17, m18 and m19.

### Example 12

### Generation of Monoclonal Antibodies to Human IL-1R by Immunization With IL-1R Bearing C127 Cells

C127 cells which express human IL-1R were prepared in order to immunize animals to the IL-1R. Briefly, Gembl, a derivative of the Embl plasmid (see Dente et al., Nucleic Acids Res. 11:1645, 1983), was first prepared by inserting the Sp6 and T7 promoter on either side of the multiple cloning site (see, for example, pGEM⁻³, ProMega Biotech, Madison, Wis.). DNA encoding human IL1R (obtained from λ9, see Sims et al., Proc Natl. Acad Sci USA 866:8946-8950, 1989) was then inserted into the EcoRI site of Gembl, creating plasmid Gembl 9A. Gembl 9A was digested with StyI, repaired with T4 polymerase, and digested with BgIII. The digestion was purified by agarose gel electrophoresis resulting in an 1863 b.p. fragment containing the sequence which encodes human IL-1R.

Trim Hixp (a derivative of pDC201, see Sims et al., Science 241:585, 1988, with some adenovirus sequences deleted) was next prepared in order to express human IL-1R from mammalian cells. Trim Hixp includes the following components: “SV40” contains SV40 sequences from coordinates 5171-270 including the origin of replication, enhancer sequences and early and late promoters. The fragment is oriented such that the direction of transcription is clockwise from the early promoter. The adenovirus Major Late Promoter (adMLP) contains adenovirus-2 sequences from pDC201, as discussed above, including the major late promoter, first exon and part of the intron between the first and second exons of the tripartite leader. The tripartite leader (TPL) contains the first exon and part of the intron between the first and second exons of the adenovirus-2 tripartite leader, the second exon and part of the third exon of the tripartite leader, and a multiple cloning site containing sites for Xho I, Kpn I, Sma I, Not I, and Bgl I. pA contains SV40 sequences from 4127-4100 and 2770-2533 that include the polyadenylation and termination signals for early transcription. Clockwise from pA are adenovirus-2 sequences 10532-11156 containing the VAI and VAII genes, followed by pBR322 sequences from 4363-2486 and 1094-375 containing the ampicillin resistance gene and origin of replication.

Trim Hixp was digested with Pvu2 and Bgm, purified, and ligated with the Gembl 9A 1863 b.p. fragment to form HFL TXPΔTPL. HFL TXPΔTPL was digested with SfiI and XmnI, and a 3610 b.p. fragment was purified. Bx8 (which contains the full length sequence for murine IL-1R, as described above) was also digested with SfiI and XmnI to produce a 10,429 b.p. fragment, and ligated to the 3610 b.p. fragment above to form BX9AΔTPL This plasmid was transfected into C127 cells (ATTC No. CRL 1616) as described above.

Mice were immunized intraperitoneally three times at three-week intervals with 10⁶ C127 cells bearing recombinant human IL-1R. All animals displayed inhibition of IL-1 binding titers ELISA titers to whole C127 cells. Two mice were IV boosted with two million cells and fused three days later. The mice were sacrificed and their spleen cells fused with P3X63 Ag8.653 mouse myelomas at a ratio of 4:1, with 50% PEG MW 1500 according to methods discussed above. Hybridomas were screened 10 days later by ELISA to C127 cells and positives were screened again two days later to both C127 cells and IL-1R negative PTP cells essentially as described below in Example 15. Among several clones, two were selected for further analysis: hIL1Rm1 and m10. Hybridoma hIL1Rm10 was deposited with the American Type Culture Collection on ______, under deposit accession number ATCC ______.

### Example 13

### Generation of Monoclonal Antibodies to Human IL-1R by Immunization With Vaccinia Bearing IL-1R

cDNA containing the entire coding region of the hIL1R was inserted into the SmaI site of the vaccinia virus (VV) plasmid coexpression vector pSC11, utilizing the method essentially described by Chakrabarti et al. in Mol. Cell Biol 5:3403-3409, 1985. pSC11 is available by license from the U.S. Department of Commerce, Nat'l Technical Information Service, 5285 Port Royal Road, Springfield, Va. 22161. Blue plaques were selected and used to infect either CV-1 (ATCC No. CCL 70) or HeLa (ATTC No. CCL 2) cells which were subsequently tested for expression of IL-1R. Recombinant VV from a positive plaque was then purified using conventional techniques (see Chakrabarti et al., supra; see also Elango et al., PNAS USA 83:1906-1910, 1986).

A Lewis rat was boosted intradermally with 10⁸ plaque forming units (pfu) of recombinant human IL-1 receptor vaccinia virus. Two weeks later the rat was boosted with 10⁶ primary rat fibroblasts infected with vaccinia virus at greater than 5 pfu per cell. Two weeks later the rat was boosted IV with 2×10⁶ C127 cells expressing recombinant human IL-1 receptor. Three days later the rat was sacrificed and its spleen cells were fused with P3×63-Ag8.653 mouse myeloma cells essentially as described above. An ELISA, as described below in Example 15, was utilized to select positive clones. One clone, hIL1Rm8, was selected for further analysis.

### Example 14

### Generation OF Polyclonal Antibodies to Murine IL-1R by Immunization With Soluble IL-1R

One hundred micrograms of soluble human IL-1R was emulsified in complete Fruend's adjuvant, and administered subcutaneously to a rabbit. Booster immunizations containing 100 μg soluble IL-1R emulsified in incomplete Fruend's adjuvant were given subcutaneously every three weeks until the rabbit's antibody titer to the IL-1R had plateaued. A final booster of 100 μg soluble IL-1R was given and the rabbit was exsanguinated 9 days later.

### Example 15

### Assays Suitable for Detecting Antibodies

Antibodies which were prepared in Examples 10 through 14 were analyzed utilizing the assays as described below. The results of these assays are presented below in Table 1.

A. ELISA Assay

Two days prior to screening, transformed C127 and non-transformed PTP cells bearing no IL-1 receptors were seeded at a concentration of 4×10⁵ cells/ml into 96-well plates to a volume of 200 μl/well. On the day of the screen, plates were washed three times with PBS and then 50 μl of hybridoma supernatants (or diluted antibody) and diluted antisera controls were added to each cell type for a 30-minute incubation at room temperature. Wells were washed three times with PBS followed by the addition of 50 μl/well of anti-species specific antisera (e.g., Goat anti-Rat Peroxidase (#172-1009) Bio-Rad) which was diluted 1:1000 in 5% fetal calf serum/PBS for 30 minutes at room temperature. Wells were washed three times with PBS and 100 μl/well of either O-Phenylenediamine(OPD) substrate solution (1 mg/ml OPD (00-2003 Zymed) and 0.001% H₂O₂ in 0.1 M citrate buffer pH 4.5) or TMB substrate (#507600 Kirkegaard and Perry) was added. Plates were read on a Titertek Multiscan Plate reader (#340 Flow Laboratories) after 15 minutes at either 450 nm (for OPD) or 650 nm (for TMB). Positives were selected as those wells having an absorbance with C127 cells three times greater than the corresponding signal generated with PmP cells.

B. ABC Assay

96-well, flat bottom polystyrene ELISA plates (Catalog #76-381-04, Linbro, McLean, Va.) were coated overnight with 10 μg/ml of goat anti-species specific IgG (Zymed, South San Francisco, Calif.). Plates were then blocked for 1 hour with 5% non-fat dry milk. One hundred ail of hybridoma supernatants (or diluted antibody) were then added for 1 hour. The plates were washed with PBS and 100 μl of iodinated soluble receptor was, added at about 2,000 cpm/μl for 1 hour. Plates were washed and exposed to film overnight. Positive spots on the film indicate the presence of precipitated protein.

C Modified ABC Assay

96 well flat bottom polystyrene ELISA plates (Catalog #76-381-04, Linbro, McLean, Va.) were coated overnight with 10 μg/ml of goat anti-species specific IgG (Zymed, South San Francisco, Calif.). Plates were then blocked for 1 hour with 5% non-fat dry milk. One hundred Al of hybridoma supernatants (or diluted antibody) were then added for 1 hour. The plates were washed with PBS and 100 μl of soluble IL-1R was added for 1 hour. The plates were again washed with PBS and 2,000 cpm/μl iodinated IL1α was added for 1 hour. Plates were then washed with PBS and exposed to film overnight. Positives indicate antibodies to the IL-1 receptor that do not inhibit the binding of IL-1 to the receptor. In contrast, a negative result indicates that the antibody bound to the IL-1 binding site of the receptor, thus inhibiting or blocking the binding of the IL-1 to the receptor.

D. Cross-Blocking Assay to Determine Epitopes

The relative epitopes of the isolated antibodies were determined by a cross-blocking assay to see if each antibody inhibits the binding of IL1 receptor to the other antibodies. Two μl of antibody or hybridoma supernatant was bound to a nitrocellulose sheet (#22060 Schleicher & Schuell), allowed to dry and blocked for 1 hour in 3% BSA in PBS. Supernatants were preincubated 1:1 with radioiodinated IL-1R at 2,000 cpm/μl for 1 hour. Two μl of the antibody receptor solution was then dotted over the bound antibody for 10 minutes. The nitrocellulose sheet was washed 3 times with PBS and exposed to film overnight. A diminished signal indicates that there is inhibition of one antibody's binding by another.

E. Dot Blot Immunoassay

A sheet of nitrocellulose membrane (Schleicher and Schuell, Keene, N.H.) is marked off into squares, and approximately 2,41 containing 25 ng of soluble murine or human IL-1 receptor is placed onto the membrane within each square. The IL-1R is allowed to dry, and then the sheet is blocked with 3% bovine serum albumin in PBS (“3% PBSA”) for one hour. The membrane is removed from the 3% PBSA, pat dry with a towel, and 2 μl of the hybridoma supernatant or antibody is then placed directly onto the membrane. After 30 minutes the membrane is washed three times quickly with PBS, followed by two 5-minute incubations in PBS to remove excess antibody. The membrane is then incubated for 30 minutes in diluted labelled antisera which is species-specific for the antibody. For example, if the hybridoma supernatant is from a mouse monoclonal, the membrane is incubated in a 1:2000 dilution of goat anti-mouse Horse Radish Peroxidase (“HRP,” Bio-Rad, Richmond, Calif.). The membrane is washed as before, and color developed by incubating the membrane in a solution containing an HRP substrate (4-chloro-1-napthol) (Kirkegaard and Perry Laboratories, Gaithersburg, Md.) and hydrogen peroxide. Positives indicate the presence of antibodies which recognize the presence of the protein which was coated onto the nitrocellulose membrane.

F. Radioimmunoprecipitation (RIP)

The following were added to a 600 μl conical tube: (1) 50 μl of PBS containing 50 mg/ml bovine serum albumin and 10 μl/ml Triton X100 (New England Nuclear, Boston, Mass.) (hereinafter referred to as “PBSTA”); (2) 2 μl of rabbit anti-species specific antibody (Le., rabbit anti mouse IgG, M, and A); (3) antibody (50 μl of hybridoma supernatant, or 2 μl of serum, or 2 μl of ascites; (4) 50 μl of 20% Protein A Sepharose solution (Protein A Sepharose CL-4B, Sigma, St. Louis, Mo.); and (5) 25 μl of radioiodinated IL-1R at about 2,000 cpm/μl. The sample is spun down for two minutes at 500 rpm in a Sorvall RT6000 centrifuge. The sample is then equilibrated overnight at 4° C. on a mini-orbital shaker (Bellco, Vineland, N.J.). After incubation overnight, about 300 μl of PBS is added to each tube, and they are centrifuged for four minutes at 500 rpm. The supernatant is aspirated and the pellet is washed twice more with PBS followed by centrifugation. Radioactivity of the pellet is measured with a gamma counter. A positive indicates the presence of an immunoprecipitating antibody.

G. Inhibition Assay

C127 cells bearing IL-1 receptors were plated into 96 well plates such that on average the cells had 250,000 IL-1 receptors per cell. Briefly, the number of receptors was calculated utilizing techniques well known in the art (see Jones et al., Molec. Immun. 16:889, 1979). Based upon the average number of receptors on these IL-1R positive cells, non-IL-1R bearing cells (ELA) (ATCC No. TIB 39) were added such that on average cells had 250,000 IL-1 receptors per cell. 2.5×10⁶ cells from this mixture were added to each well of the plate.

Antibody was added to the wells starting at 100 μg/ml followed by threefold serial dilutions. ¹²⁵I-IL-1α was then added to each well, and the plate was incubated for 2 hours at 4° C. with gentle shaking. The cells and antibody were then placed into microfuge tubes containing thalate oil, and spun in a microfuge (see Jones et al., supra). This separates bound from unbound antibody. The microfuge tube was then cut in half to separate the bound antibody (pelleted cells) from the unbound antibody (remaining in suspension). The pellet was counted in a gamma counter to determine the presence of ¹²⁵I. Presence of ¹²⁵I indicates that the antibody does not inhibit the binding of IL1-1 to the receptor.

H. Isotyping

Isotype of monoclonals was determined utilizing either an MonoAB-ID EIA rat isotyping kit (#93-9550 Zymed), or with a Hyclone mouse isotyping kit (Logan, Utah) according to the manufacturer's instructions.

TABLE 1
Characterization of antibodies

| | | | Modified | Dot | | Inhi- | |
|---|---|---|---|---|---|---|---|
| Antibody | ELISA | ABC | ABC | Blot | RIP | bition | Isotype |
| mIL1Rm1, | + | + | + | weak | + | − | IgG₂ₐ |
| m3, m5 | | | | | | | (rat) |
| mIL1Rm15 | + | + | − | + | + | + | IgG₂ₐ |
| | | | | | | | (rat) |
| mIL1Rm16 | + | + | + | + | + | − | IgG₂ₐ |
| m17, m18 | | | | | | | (rat) |
| mIL1Rm19 | + | + | + | + | + | − | IgG_{2b} |
| | | | | | | | (rat) |
| hIL1Rm1, | + | + | − | + | + | + | IgG₁ |
| m10 | | | | | | | (mouse) |
| hIL1Rm8, | + | + | + | + | + | − | IgG_{2b} |
| | | | | | | | (rat) |

### Example 16

### Production and Purification of Antibodies

A. Production of Antibodies in Rats

Lewis rats were first primed with 0.5 ml of pristane (2,4,6,10 tetramethylpentadecane, Aldrich, Milwaukee, Wis.). Two weeks later 1×10⁶ rat hybridomas (e.g., cell line mIL1Rm15) in PBS were injected intraperitoneally into the rat. Approximately two to five weeks later ascites fluid was removed from the rat, and centrifuged to remove cells and particulate matter.

B. Purification on Protein G

Two milliliters of ascites fluid was applied to a 1 ml column of protein G sepharose (Pharmacia, Piscataway, N.J.) diluted 1:1 with 0.1 M sodium acetate pH 4.5. The column was washed with 15 column volumes of sodium acetate pH 4.5. Purified antibody was then eluted with 0.1 M glycine HCl pH 3.0 and neutralized with 2 M TRIS.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A monoclonal antibody which specifically binds to a mammalian IL-1 receptor.

2. A monoclonal antibody according to claim 1, selected from the group consisting of murine, rat and human monoclonal antibodies.

3. A monoclonal antibody according to claim 2 which specifically binds to a human IL-1 receptor.

4. A murine monoclonal antibody according to claim 3.

5. A human monoclonal antibody according to claim 3.

6. A murine monoclonal antibody according to claim 4 which blocks the binding of human IL-1 to human IL-1 receptor.

7. A human monoclonal antibody according to claim 5 which blocks the binding of human IL-1 to human IL-1 receptor.

8. A murine monoclonal antibody according to claim 6, hIL1Rm10.

9. A murine hybridoma which produces a monoclonal antibody according to claim 8, ATCC ______.

10. A monoclonal antibody according to claim 2 which specifically binds to a murine IL-1 receptor.

11. A rat monoclonal antibody according to claim 10.

12. A therapeutic composition comprising a monoclonal antibody to the IL-1 receptor according to claim 7, and a physiologically acceptable carrier or diluent.

13. A binding protein which specifically binds to a mammalian IL-1 receptor comprising an IL-1 receptor-binding domain encoded by a DNA sequence encoding an antibody or portion thereof which specifically binds to a mammalian IL-1 receptor.

14. A therapeutic composition comprising a binding protein according to claim 13 which specifically binds to mammalian IL-1 receptor, and a physiologically acceptable carrier or diluent.

15. A method for detecting IL-1 receptors on cells, comprising:
(a) incubating the cells with a labeled monoclonal antibody according to claim 1; and
(b) detecting the presence of bound antibody.

16. A method for detecting soluble IL-1 receptor in serum, comprising:
(a) incubating serum suspected of containing soluble IL1 receptor with a solid support having monoclonal antibodies according to claim 1 affixed thereto under conditions and for a time sufficient for binding to occur;
(b) incubating the solid support with a second labeled antibody specific for mammalian IL-1 receptors under conditions and for a time sufficient for binding to occur; and
(c) detecting the presence of bound labeled antibody.
